(19) United States
(12) Patent Application Publication
(10) Pub. No.: US 2010/0093726 A1
(43) Pub. Date: Apr. 15, 2010

(54) NOVEL 4-AMINO-QUINOLINE DERIVATIVES USEFUL AS ANTI-MALARIA DRUGS

(75) Inventors: Giuseppe Campiani, Chianciano Terme (IT); Sandra Gemma, Martignano (IT); Caterina Fattorusso, Napoli (IT); Gagan Kukreja, Siena (IT); Bhupendra Prasad Joshi, Siena (IT); Stefania Butini, Siena (IT); Marco Persico, Sorrento (IT); Salvator Sanna Coccone, Onano (IT); Matteo Bernetti, Montepulciano (IT)

Correspondence Address: BIRCH STEWART KOLASCH & BIRCH, PO BOX 747, FALLS CHURCH, VA, 22040-0747, US

(73) Assignee: Giuseppe CAMPIANI, Chianciano Teme (IT)

(21) Appl. No.: 12/527,454

(22) PCT Filed: Feb. 18, 2008
(86) PCT No.: PCT/EP2008/051923

§ 371 Date: Oct. 8, 2009

(30) Foreign Application Priority Data
Feb. 20, 2007 (DK) PA 2007 00263

(51) Int. Cl. C07D 215/44; A61K 31/47; A61K 31/4725; A61K 31/5377; C07D 413/12; C07D 401/12; A61K 31/496; A61P 33/00; A61P 33/06

(52) U.S. Cl. 514/235.2; 546/160; 514/313; 544/128; 544/363; 514/253.06

(57) ABSTRACT

The present invention relates to clotrimazole/quinoline hybrids useful as active ingredients of anti-malaria drugs. The compounds show a remarkable in vitro biological activity especially against the chloroquine-resistant Plasmodium falciparum strains and in vivo activity against P. berghei.

### TECHNICAL FIELD

The present invention relates to clotrimazole/quinoline hybrids useful as active ingredients of anti-malaria drugs. The compounds show a remarkable in vitro biological activity especially against the chloroquine-resistant Plasmodium falciparum strains and in vivo activity against P. berghei.

### BACKGROUND ART

Malaria, a disease caused by the parasite Plasmodium falciparum (Pf), represents one of the most widespread and deadly parasitic disease in man. For over 50 years, chloroquine (CQ) and related 4-aminoquinolines (amodiaquine, mefloquine, etc) have been the drugs of choice for malaria chemotherapy, until their efficacy was hampered by the spreading of CQ-resistant (CQ-R) Pf strains. Recently the amodiaquine regioisomer isoquine has been developed and is currently under investigation.

Although four species of the genus Plasmodium cause human malaria, most fatal malaria cases are due to infection by Pf which has a widespread geographic distribution and is most likely to be drug resistant. The parasites are transmitted through the bite of mosquitoes of the genus Anopheles. After infection of humans, Pf establishes an initial asymptomatic stage in liver cells followed by an erythrocytic stage which is responsible for the clinical symptoms of the disease. During the intra-erythrocytic stage of Pf life cycle, the catabolism of hemoglobin (Hb) is a key source of food for the parasite. Hb proteolysis occurs within the acidic (pH 4.9-5.4) digestive vacuole generating amino acids and releasing toxic free heme (ferriprotoporphyrin IX, FP). This latter is detoxified by the Plasmodium through polymerisation into an inert crystal known as the malaria pigment hemozoin. A second detoxification pathway is diffusion of free heme through the vacuolar membrane to the cytoplasm of the parasite and degradation by reduced glutathione.

The main mode of action of CQ is based on its ability to accumulate into the acidic Pf food vacuole (FV) and to interfere with the detoxification of free heme.

Studies have demonstrated that chlotrimazole (CLT), a potent anti-mycotic drug which act through inhibition of fungal P450 cytochrome 14α-lanosterol demethylase, shows moderate in vitro antimalarial activity against different CQ-resistant (CQ-R) and CQ-sensitive (CQ-S) strains of the malaria parasite Pf. The imidazole moiety of CLT behaves as an heme axial ligand which form stable, bulky and hydrophobic heme-CLT complex thereby enhancing the colloid-osmotic hemolysis and thus preventing heme from β-hematin formation, eventually disturbing the hemoglobin catabolism in the malarial parasite. In addition CLT seems to be involved in the inhibition of glutathione dependent heme degradation, resulting in enhancement of heme-induced hemolysis, thus damaging the cell membrane more than the free heme itself. Moreover, it has been suggested that in the presence of H₂O₂, CLT inhibits Pf hemoperoxidase. It is believed that this enzyme could be involved in the detoxification of H₂O₂.

Even though the in vitro antimalarial properties of CLT are interesting and it may represent a new hit in the field, our studies indicated that CLT is not active against P. berghei in vivo at doses up to 100 mg/Kg, making the use of CLT in antimalarial therapy not viable.

Gemma et al.; Bioorganic & Medicinal Chemistry Letters 2006 16 5384-5388, describe the synthesis of a series of N1-arylidene-N2-quinolyl- and N2-acrydinylhydrazones, useful as antimalarial agents. However, the 4-amino-quinoline derivatives of the present invention are not described.

Gemma et al.; Journal of Medicinal Chemistry 2007 50 595-598, describe a polycyclic pharmacophore related to clotrimazole, and its antimalarial activity. However, the 4-amino-quinoline derivatives of the present invention are not described.

### SUMMARY OF THE INVENTION

Starting from the interesting antimalarial properties of CLT, and taking also into account the mechanism of action of 4-aminoquinoline antimalarials like CQ, isoquine and amodiaquine, we designed and synthesized a series of CLT/quinoline hybrid compounds based on a novel pharmacophore with the aim to ameliorate the in vitro activity against CQ-S and CQ-R strains with respect to CLT and CQ, to overcome resistance to CQ and to obtain compounds active in vivo against the more common animal models of malaria.

The compounds of the invention present a remarkable in vitro biological activity especially against the CQ-R Pf strains, much higher than that of CLT and CQ, and a good antimalarial activity in vivo against P. berghei (ED₅₀=6.3 mg/Kg) which is promising for the development of new antimalarial drugs. Moreover, synthesis of the compounds of the invention is characterized by low costs of production which addresses to the economic burden associated with this orphan disease.

The compounds of the invention may be described as 4-amino-quinoline derivatives represented by Formula I,

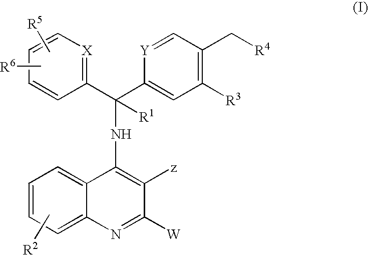

an isomer thereof or a mixture of its isomers, or a pharmaceutically acceptable salt thereof, wherein

X and Y both represent CH; or X and Y, together with the carbon atoms to which they are attached, form a bridge selected from C—C, C—CH₂—CH₂—C and C—CH═CH—C;

W and Z both represent hydrogen; or W and Z, together with the carbon atoms to which they are attached, form a benzo-fused ring —CH═CH—CH═CH—, which fused ring may optionally be substituted with halo;

R¹ represents hydrogen, phenyl, halo-substituted phenyl, 3,4-methylendioxyphenyl or (pyrrolidinylmethyl)phenyl;

R² represents halo, trifluoromethyl or alkoxy;

R³ represents hydrogen, halo, hydroxy, cyano, sulfonamido or dialkylsulfonamido;

R⁴ represents N,N-dialkyl-amino, pyrrolidinyl, piperazinyl, morpholinyl or imidazolyl;

R⁵ represents hydrogen, halo, cyano, hydroxy or —SO₂NH₂; and

R⁶ represents hydrogen, N,N-dialkyl-amino-methyl, pyrrolidinyl-methyl, piperazinyl-methyl, morpholinyl-methyl or 1H-imidazolyl-methyl;

as described in more details below.

In another aspect the invention provides pharmaceutical compositions comprising a therapeutically effective amount of a 4-amino-quinoline derivative of the invention, or a pharmaceutically-acceptable addition salt thereof, or a prodrug thereof, together with one or more adjuvants, excipients, carriers and/or diluents.

Viewed from another third aspect the invention relates to the use of the 4-amino-quinoline derivative of the invention, or a pharmaceutically-acceptable addition salt thereof, for the manufacture of a pharmaceutical composition/medicament.

In a further aspect the invention provides a method of treatment, prevention or alleviation of an infectious disease or a disorder or a condition of a living animal body, including a human, which disorder, disease or condition is caused by a parasite of the genus Plasmodium, which method comprises the step of administering to such a living animal body in need thereof, a therapeutically effective amount of the 4-amino-quinoline derivative of the invention, or a pharmaceutically-acceptable addition salt thereof, or a prodrug thereof.

Other objects of the invention will be apparent to the person skilled in the art from the following detailed description and examples.

### DETAILED DISCLOSURE OF THE INVENTION

In its first aspect the invention provides a 4-amino-quinoline derivative represented by Formula I,

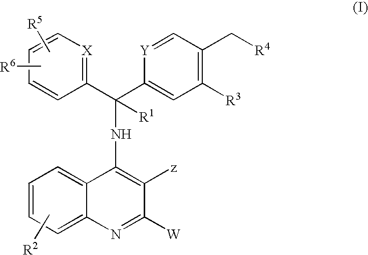

an isomer thereof or a mixture of its isomers, or a pharmaceutically acceptable salt thereof, wherein

X and Y both represent CH; or X and Y, together with the carbon atoms to which they are attached, form a bridge selected from C—C, C—CH₂—CH₂—C and C—CH═CH—C;

W and Z both represent hydrogen; or W and Z, together with the carbon atoms to which they are attached, form a benzo-fused ring —CH═CH—CH═CH—, which fused ring may optionally be substituted with halo;

R¹ represents hydrogen, phenyl, halo-substituted phenyl, 3,4-methylendioxyphenyl or (pyrrolidinylmethyl)phenyl;

R² represents halo, trifluoromethyl or alkoxy;

R³ represents hydrogen, halo, hydroxy, cyano, sulfonamido or dialkylsulfonamido;

R⁴ represents N,N-dialkyl-amino, pyrrolidinyl, piperazinyl, morpholinyl or imidazolyl;

R⁵ represents hydrogen, halo, cyano, hydroxy or —SO₂NH₂; and

R⁶ represents hydrogen, N,N-dialkyl-amino-methyl, pyrrolidinyl-methyl, piperazinyl-methyl, morpholinyl-methyl or 1H-imidazolyl-methyl.

In a preferred embodiment, however,

if R₂ represents halo, trifluoromethyl or alkoxy; X and Y both represent CH; and W and Z both represent hydrogen;

then the 4-amino-quinoline derivative of the invention is a compound of Formula I wherein

R₁ represents hydrogen, phenyl, fluorophenyl, 3,4-methylendioxyphenyl or (pyrrolidinylmethyl)phenyl;

R₃ represents hydrogen, halo or hydroxy;

R₄ represents N,N-dialkyl-amino, pyrrolidinyl, piperazynyl, morpholinyl or imidazolyl;

R₅ represents hydrogen, halo, cyano, hydroxy or SO₂NH₂; and

R₆ represents hydrogen, N,N-dialkyl-aminomethyl, pyrrolidinylmethyl, piperazynylmethyl, morpholinylmethyl or imidazolylmethyl.

In another preferred embodiment, however,

if R₂ represents alkoxy; and X and Y both represent CH;

then the 4-amino-quinoline derivative of the invention is a compound of Formula I wherein

R₁ represents hydrogen, phenyl, fluorophenyl, 3,4-methylendioxyphenyl or (pyrrolidinylmethyl)phenyl;

R₃ represents hydrogen, halo or hydroxy;

R₄ represents N,N-dialkyl-amino, pyrrolidinyl, piperazynyl, morpholinyl or imidazolyl;

R₅ represents hydrogen, halo, cyano, hydroxy or SO₂NH₂;

R₆ represents hydrogen, N,N-dialkyl-aminomethyl, pyrrolidinylmethyl, piperazynylmethyl, morpholinylmethyl or imidazolylmethyl; and

W and Z, together with the carbon atoms to which they are attached, form a benzo-fused ring —CH═CH—CH═CH—, which fused ring may optionally be substituted with halo.

In a third preferred embodiment, however,

if R₂ represents halo, trifluoromethyl or alkoxy; X and Y, together with the carbon atoms to which they are attached, form a bridge selected from C—C, C—CH₂—CH₂—C and C—CH═CH—C; and R₁ represents hydrogen;

then the 4-amino-quinoline derivative of the invention is a compound of Formula I wherein W and Z both represent hydrogen; or

W and Z, together with the carbon atoms to which they are attached, form a benzo-fused ring —CH═CH—CH═CH—, which fused ring may optionally be substituted with halo;

R₃ represents hydrogen, halo or hydroxy;

R₄ represents N,N-dialkyl-amino, pyrrolidinyl, piperazynyl, morpholinyl or imidazolyl;

R₅ represents hydrogen, halo or hydroxy; and

R₆ represents hydrogen, N,N-dialkyl-aminomethyl, pyrrolidinylmethyl, piperazynylmethyl, morpholinylmethyl or imidazolylmethyl.

In a more preferred embodiment the 4-amino-quinoline derivative of the invention is a compound of Formula I, wherein

X and Y, together with the carbon atoms to which they are attached, form a bridge selected from C—C, C—CH₂—CH₂—C and C—CH═CH—C; and

W and Z both represent hydrogen; or

W and Z, together with the carbon atoms to which they are attached, form a benzo-fused ring —CH═CH—CH═CH—, which fused ring may optionally be substituted with halo;

R₁ represents hydrogen;

R₂ represents halo, trifluoromethyl or alkoxy;

R₃ represents hydrogen, halo or hydroxy;

R₄ represents N,N-dialkyl-amino, pyrrolidinyl, piperazynyl, morpholinyl or imidazolyl;

R₅ represents hydrogen, halo or hydroxy; and

R₆ represents hydrogen, N,N-dialkyl-aminomethyl, pyrrolidinylmethyl, piperazynylmethyl, morpholinylmethyl or imidazolylmethyl.

In a fourth preferred embodiment the 4-amino-quinoline derivative of the invention is a compound of Formula I, wherein X and Y both represent CH.

In a fifth preferred embodiment the 4-amino-quinoline derivative of the invention is a compound of Formula I, wherein X and Y, together with the carbon atoms to which they are attached, form a bridge selected from C—C, C—CH₂—CH₂—C and C—CH═CH—C.

In a more preferred embodiment X and Y, together with the carbon atoms to which they are attached, form a C—C bridge.

In another more preferred embodiment X and Y, together with the carbon atoms to which they are attached, form a C—CH₂—CH₂—C bridge.

In a third more preferred embodiment X and Y, together with the carbon atoms to which they are attached, form a C—CH═CH—C bridge.

In a sixth preferred embodiment the 4-amino-quinoline derivative of the invention is a compound of Formula I, wherein W and Z both represent hydrogen.

In a seventh preferred embodiment the 4-amino-quinoline derivative of the invention is a compound of Formula I, wherein W and Z, together with the carbon atoms to which they are attached, form a benzo-fused ring —CH═CH—CH═CH—, which fused ring may optionally be substituted with halo.

In an eighth preferred embodiment the 4-amino-quinoline derivative of the invention is a compound of Formula I, wherein W and Z, together with the carbon atoms to which they are attached, form a benzo-fused ring selected from —CH═CH—CH═CH— and —CH═CH—C(Cl)═CH—.

In a more preferred embodiment W and Z, together with the carbon atoms to which they are attached, form a —CH═CH—C(Cl)═CH— benzo-fused ring.

In a ninth preferred embodiment the 4-amino-quinoline derivative of the invention is a compound of Formula I, wherein

X and Y both represent CH;

W and Z both represent hydrogen;

R₁ represents hydrogen, phenyl, fluorophenyl, 3,4-methylendioxyphenyl or (pyrrolidinylmethyl)phenyl;

R₂ represents halo, trifluoromethyl or alkoxy;

R₃ represents hydrogen, halo, hydroxy, cyano, sulfonamido or dialkylsulfonamido;

R₄ represents N,N-dialkyl-amino, pyrrolidinyl, piperazynyl, morpholinyl or imidazolyl;

R₅ represents hydrogen, halo, cyano, hydroxy or SO₂NH₂; and

R₆ represents hydrogen, N,N-dialkyl-aminomethyl, pyrrolidinylmethyl, piperazynylmethyl, morpholinylmethyl or imidazolylmethyl.

In a more preferred embodiment

X and Y both represent CH;

W and Z both represent hydrogen;

R₁ represents hydrogen, phenyl, fluorophenyl, 3,4-methylendioxyphenyl or (pyrrolidinylmethyl)phenyl;

R₂ represents halo, trifluoromethyl or alkoxy;

R₃ represents hydrogen, halo or hydroxy;

R₄ represents N,N-dialkyl-amino, pyrrolidinyl, piperazynyl, morpholinyl or imidazolyl;

R₅ represents hydrogen, halo, cyano, hydroxy or SO₂NH₂; and

R₆ represents hydrogen, N,N-dialkyl-aminomethyl, pyrrolidinylmethyl, piperazynylmethyl, morpholinylmethyl or imidazolylmethyl.

In another more preferred embodiment R₁ represents hydrogen, phenyl, p-fluorophenyl, 3,4-methylendioxyphenyl or p-[(pyrrolidin-1-yl)methyl]phenyl.

In an even more preferred embodiment R₁ represents hydrogen, phenyl or p-fluorophenyl.

In a third more preferred embodiment R₂ represents 7-halo, in particular 7-chloro, 7-trifluoromethyl or 6-alkoxy, in particular 6-methoxy.

In a fourth more preferred embodiment R₃ represents hydrogen, chloro, fluoro or hydroxy.

In an even more preferred embodiment R₃ represents hydrogen.

In a fifth more preferred embodiment R₄ represents N,N-di-C₁₋₄-alkyl-amino, in particular N,N-dimethyl-amino or N,N-diethyl-amino, pyrrolidin-1-yl, piperazyn-1-yl, morpholin-4-yl or 1H-imidazol-1-yl.

In an even more preferred embodiment R₄ represents pyrrolidin-1-yl or morpholin-4-yl.

In a sixth more preferred embodiment R₅ represents hydrogen, fluoro, chloro, cyano, hydroxy or SO₂NH₂.

In an even more preferred embodiment R₅ represents halo, and in particular chloro.

In a seventh more preferred embodiment R₆ represents hydrogen, N,N-di-C₁₋₄-alkyl-aminomethyl, in particular N,N-dimethyl-aminomethyl or N,N-diethyl-aminomethyl, pyrrolidin-1-ylmethyl, piperazyn-1-ylmethyl, morpholin-4-ylmethyl or 1H-imidazol-1-ylmethyl.

In an even more preferred embodiment R₆ represents hydrogen.

In yet another more preferred embodiment

X and Y both represent CH;

W and Z both represent hydrogen;

R₁ represents hydrogen, phenyl or fluorophenyl;

R₂ represents halo, trifluoromethyl or alkoxy;

R₃ represents hydrogen;

R₄ represents pyrrolidinyl or morpholinyl;

R₅ represents halo; and

R₆ represents hydrogen.

In an eight preferred embodiment the 4-amino-quinoline derivative of the invention is a compound of Formula I, wherein

X and Y both represent CH;

W and Z, together with the carbon atoms to which they are attached, form a benzo-fused ring —CH═CH—CH═CH—, which fused ring may optionally be substituted with halo;

R₁ represents hydrogen, phenyl, fluorophenyl, 3,4-methylendioxyphenyl or (pyrrolidinylmethyl)phenyl;

R₂ represents alkoxy;

R₃ represents hydrogen, halo, or hydroxy, cyano, sulfonamido or dialkylsulfonamido;

R₄ represents N,N-dialkyl-amino, pyrrolidinyl, piperazynyl, morpholinyl or imidazolyl;

R₅ represents hydrogen, halo, cyano, hydroxy or SO₂NH₂; and

R₆ represents hydrogen, N,N-dialkyl-aminomethyl, pyrrolidinylmethyl, piperazynylmethyl, morpholinylmethyl or imidazolylmethyl.

In a more preferred embodiment

X and Y both represent CH;

W and Z, together with the carbon atoms to which they are attached, form a benzo-fused ring —CH═CH—CH═CH—, which fused ring may optionally be substituted with halo;

R₁ represents hydrogen, phenyl, fluorophenyl, 3,4-methylendioxyphenyl or (pyrrolidinylmethyl)phenyl;

R₂ represents alkoxy;

R₃ represents hydrogen, halo or hydroxy;

R₄ represents N,N-dialkyl-amino, pyrrolidinyl, piperazynyl, morpholinyl or imidazolyl;

R₅ represents hydrogen, halo, cyano, hydroxy or SO₂NH₂; and

R₆ represents hydrogen, N,N-dialkyl-aminomethyl, pyrrolidinylmethyl, piperazynylmethyl, morpholinylmethyl or imidazolylmethyl.

In another more preferred embodiment R₁ represents hydrogen, phenyl, p-fluorophenyl, 3,4-methylendioxyphenyl or p-[(pyrrolidin-1-yl)methyl]phenyl.

In an even more preferred embodiment R₁ represents hydrogen.

In a third more preferred embodiment R₂ represents methoxy, and in particular 6-methoxy.

In a fourth more preferred embodiment R₃ represents hydrogen, chloro, fluoro or hydroxy.

In an even more preferred embodiment R₃ represents hydrogen.

In a fifth more preferred embodiment R₄ represents N,N-di-C₁₋₄-alkyl-amino, in particular N,N-dimethyl-amino or N,N-diethyl-amino, pyrrolidin-1-yl, piperazyn-1-yl, morpholin-4-yl or 1H-imidazol-1-yl.

In an even more preferred embodiment R₄ represents pyrrolidin-1-yl or morpholin-4-yl.

In a sixth more preferred embodiment R₅ represents hydrogen, fluoro, chloro, cyano, hydroxy or SO₂NH₂.

In an even more preferred embodiment R₅ represents halo, and in particular chloro.

In a seventh more preferred embodiment R₆ represents hydrogen, N,N-di-C₁₋₄-alkyl-aminomethyl, in particular N,N-dimethyl-aminomethyl or N,N-diethyl-aminomethyl, pyrrolidin-1-ylmethyl, piperazyn-1-ylmethyl, morpholin-4-ylmethyl or 1H-imidazol-1-ylmethyl.

In an even more preferred embodiment R₆ represents hydrogen.

In another more preferred embodiment

X and Y both represent CH;

W and Z, together with the carbon atoms to which they are attached, form a benzo-fused ring —CH═CH—CH═CH—, which fused ring may optionally be substituted with halo;

R₁ represents hydrogen, phenyl or fluorophenyl;

R₂ represents alkoxy;

R₃ represents hydrogen;

R₄ represents pyrrolidinyl or morpholinyl;

R₅ represents halo; and

R₆ represents hydrogen.

In still another more preferred embodiment

X and Y both represent CH;

W and Z, together with the carbon atoms to which they are attached, form a benzo-fused ring —CH═CH—CH═CH—, which fused ring may optionally be substituted with halo;

R₁ represents hydrogen;

R₂ represents alkoxy;

R₃ represents hydrogen;

R₄ represents pyrrolidinyl or morpholinyl;

R₅ represents halo; and

R₆ represents hydrogen.

In an eight preferred embodiment the 4-amino-quinoline derivative of the invention is a compound of Formula I, wherein

X and Y, together with the carbon atoms to which they are attached, form a bridge selected from C—C, C—CH₂—CH₂—C and C—CH═CH—C; and

W and Z both represent hydrogen; or

W and Z, together with the carbon atoms to which they are attached, form a benzo-fused ring —CH═CH—CH═CH—, which fused ring may optionally be substituted with halo;

R₁ represents hydrogen;

R₂ represents halo, trifluoromethyl or alkoxy;

R₃ represents hydrogen, halo or hydroxy;

R₄ represents N,N-dialkyl-amino, pyrrolidinyl, piperazynyl, morpholinyl or imidazolyl;

R₅ represents hydrogen, halo or hydroxy; and

R₆ represents hydrogen, N,N-dialkyl-aminomethyl, pyrrolidinylmethyl, piperazynylmethyl, morpholinylmethyl or imidazolylmethyl.

In a more preferred embodiment R₂ represents halo, in particular 7-halo, trifluoromethyl in particular 7-trifluoromethyl or alkoxy, in particular methoxy.

In an even more preferred embodiment R₂ represents 7-halo, in particular 7-chloro, 7-trifluoromethyl or 6-alkoxy, in particular 6-methoxy.

In another more preferred embodiment R₃ represents hydrogen, fluoro, chloro or hydroxy.

In a third more preferred embodiment R₄ represents N,N-di-C₁₋₄-alkyl-amino, in particular N,N-dimethyl-amino or N,N-diethyl-amino, pyrrolidin-1-yl, piperazyn-1-yl, morpholin-4-yl or 1H-imidazol-1-yl.

In a fourth more preferred embodiment R₅ represents hydrogen, fluoro, chloro or hydroxy.

In a fifth more preferred embodiment R₆ represents hydrogen, N,N-di-C₁₋₄-alkyl-aminomethyl, in particular N,N-dimethyl-aminomethyl or N,N-diethyl-aminomethyl, pyrrolidin-1-ylmethyl, piperazyn-1-ylmethyl, morpholin-4-ylmethyl or 1H-imidazol-1-ylmethyl.

In a most preferred embodiment the 4-amino-quinoline derivative of the invention is
(±)-7-Chloro-N-{(3-chlorophenyl)[4-(pyrrolidin-1-ylmethyl)phenyl]methyl}-4-aminoquinoline (9a);
(±)-7-Chloro-N-{(3-chlorophenyl)[4-(morpholin-4-ylmethyl)phenyl]methyl}-4-aminoquinoline (9b);
(±)-7-Chloro-N-{[3-chloro-4-(pyrrolidin-1-ylmethyl)phenyl](3-chlorophenyl)methyl}-4-aminoquinoline (9c);
(±)-7-Chloro-N-{[3-chloro-4-(morpholin-4-ylmethyl)phenyl](3-chlorophenyl)methyl}-4-aminoquinoline (9d);
(±)-7-Chloro-N-{(4-chlorophenyl)[4-(pyrrolidin-1-ylmethyl)phenyl]methyl}-4-aminoquinoline (9e);
(±)-7-Chloro-N-{(4-chlorophenyl)[4-(morpholin-4-ylmethyl)phenyl]methyl}-4-aminoquinoline (9f);
(±)-7-Chloro-N-{(4-chlorophenyl)[4-(piperazin-1-ylmethyl)phenyl]methyl}-4-aminoquinoline (9g);
(±)-7-Chloro-N-{(4-chlorophenyl)[3-fluoro-4-(pyrrolidin-1-ylmethyl)phenyl]methyl}-4-aminoquinoline (9h);
(±)-N-{[3-Chloro-4-(pyrrolidin-1-ylmethyl)phenyl](3-chlorophenyl)methyl}-7-trifluoromethyl-4-aminoquinoline (9i);
(±)-N-{[3-Chloro-4-(morpholin-4-ylmethyl)phenyl](3-chlorophenyl)methyl}-7-trifluoromethyl-4-aminoquinoline (9j);
(±)-N-{(4-Chlorophenyl)[4-(pyrrolidin-1-ylmethyl)phenyl]methyl}-7-trifluoromethyl-4-aminoquinoline (9k);
(±)-N-{(4-Chlorophenyl)[3-fluoro-4-(pyrrolidin-1-ylmethyl)phenyl]methyl}-7-trifluoromethyl-4-aminoquinoline (9l);
(±)-6-Methoxy-N-{(3-chlorophenyl)[4-(pyrrolidin-1-ylmethyl)phenyl]methyl}-4-aminoquinoline (9m);
(±)-N-{(3-Chlorophenyl)[4-(morpholin-4-ylmethyl)phenyl]methyl}-6-methoxy-4-aminoquinoline (9n);
(±)-N-{[3-Chloro-4-(pyrrolidin-1-ylmethyl)phenyl](3-chlorophenyl)methyl}-6-methoxy-4-aminoquinoline (9o);
(±)-6-Methoxy-N-{(4-chlorophenyl)[4-(pyrrolidin-1-ylmethyl)phenyl]methyl}-4-aminoquinoline (9p);
(±)-N-{(4-Chlorophenyl)[4-(morpholin-4-ylmethyl)phenyl]methyl}-6-methoxy-4-aminoquinoline (9q);
(±)-N-{(4-Chlorophenyl)[4-(piperazin-1-ylmethyl)phenyl]methyl}-6-methoxy-4-aminoquinoline (9r);
(±)-6-Chloro-N-{(4-chlorophenyl)[4-(pyrrolidin-1-ylmethyl)phenyl]methyl}-2-methoxy-9-aminoacridine (9s);
(±)-7-Chloro-N-{[4-(1H-imidazol-1-yl)methylphenyl](4-chlorophenyl)methyl}-4-aminoquinoline (9t);
(±)-7-Chloro-N-{(4-chlorophenyl)[4-(pyrrolidin-1-ylmethyl)phenyl]phenyl methyl}-4-aminoquinoline (15a);
(±)-7-Chloro-N-{(4-chlorophenyl)(4-fluorophenyl)[4-(pyrrolidin-1-ylmethyl)phenyl]methyl}-4-aminoquinoline (15b);
(±)-N-{(4-Chlorophenyl)[4-(pyrrolidin-1-ylmethyl)phenyl]phenylmethyl}-6-chloro-2-methoxy-9-aminoacridine (15c); or
N-{bis[3-Chloro-4-(pyrrolidin-1-ylmethyl)phenyl]phenylmethyl}-7-chloro-4-aminoquinoline (15d);

or a pharmaceutically acceptable addition salt thereof.

Any combination of two or more of the embodiments described herein is considered within the scope of the present invention.

### Definition of Substituents

In the context of this invention halo represents fluoro, chloro, bromo or iodo. Fluoro and chloro represent preferred halo atoms of the invention.

In the context of this invention an alkyl group designates a univalent saturated, straight or branched hydrocarbon chain. The hydrocarbon chain preferably contain of from one to eighteen carbon atoms (C₁₋₁₈-alkyl), more preferred of from one to six carbon atoms (C₁₋₆-alkyl; lower alkyl), including pentyl, isopentyl, neopentyl, tertiary pentyl, hexyl and isohexyl. In a preferred embodiment alkyl represents a C₁₋₄-alkyl group, including butyl, isobutyl, secondary butyl and tertiary butyl. In another preferred embodiment of this invention alkyl represents a C₁₋₃-alkyl group, which may in particular be methyl, ethyl, propyl or isopropyl.

In the context of this invention an alkoxy group designates an “alkyl-O-” group, wherein alkyl is as defined above. Examples of preferred alkoxy groups of the invention include methoxy and ethoxy.

### Pharmaceutically Acceptable Salts

The 4-amino-quinoline derivatives of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysine, and the ammonium salt, and the like, of a chemical compound of the invention containing an anionic group. Such cationic salts may be formed by procedures well known and described in the art.

### Isomers

It will be appreciated by those skilled in the art that the 4-amino-quinoline derivatives of the present invention may exist in different stereoisomeric forms, including enantiomers, diastereomers, as well as geometric isomers (cis-trans isomers). The invention includes all such isomers and any mixtures thereof including racemic mixtures.

Methods for the resolving the optical isomers are known in the art. Such methods include those described by Jaques J, Collet A, & Wilen S in “Enantiomers, Racemates and Resolutions”, John Wiley and Sons, New York (1981).

Optical active compounds can also be prepared from optically active starting materials or intermediates.

### Methods of Preparation

The 4-amino-quinoline derivatives of the invention may be prepared by conventional methods for chemical synthesis, e.g. those described in the working examples. The starting materials for the processes described in the present application are known or may readily be prepared by conventional methods from commercially available chemicals.

The 4-amino-quinoline derivatives may be prepared from readily available starting materials by following general methods and procedures. It will be appreciated that where typical or preferred experimental conditions (i.e. reaction temperatures, time, moles of reagents, solvents, etc.) are given, other experimental conditions can also be used, unless otherwise stated. Optimum reaction conditions may vary with the particular reactants or solvents used, but such conditions can be determined by one skilled in the art by routine optimization procedures. Specific reference is made to the methods described in the Examples and to the Schemes 1-2.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

The compounds of the invention present a remarkable in vitro biological activity especially against the CQ-R Pf strains, much higher than that of CLT and CQ, and a good antimalarial activity in vivo against P. berghei which is promising for the development of new antimalarial drugs.

Preliminary evaluation of the putative mechanism of action of the compounds showed that interference with heme polymerisation and detoxification processes could be involved as in vitro inhibition of β-hematin formation was observed. Moreover, in contrast to CLT, the compounds of the invention do not interact with heme in P450 cytochromes as demonstrated by their lack of anti-fungal activity being selective for free heme. Free heme represents a valuable target in the design of new antimalarial agents as the lack of interaction with a specific protein target can decrease the potential of inducing resistance under drug pressure.

Therefore, in another aspect the invention relates to use of the 4-amino-quinoline derivatives of the invention, or a pharmaceutically-acceptable addition salt thereof, or a prodrug thereof, for the manufacture of a pharmaceutical composition/medicament for the treatment, prevention or alleviation of an infectious disease, disorder or condition of a mammal, including a human, which disease, disorder or condition is caused by a parasite of the genus Plasmodium.

In a more preferred embodiment the disease, disorder or condition is caused by P. falciparum, P. berghei, P. vivax, P. ovale, P. malaria or P. knowlesi.

In a most preferred embodiment invention relates to use of the 4-amino-quinoline derivatives of the invention for the treatment, prevention or alleviation of malaria. Such use includes the treatment of a mammal suffering from malaria infection or at risk of being infected.

The term “therapeutically effective amount” as used herein refers to an amount of a therapeutic agent needed to treat, ameliorate a targeted disease or condition, or to exhibit a detectable therapeutic effect.

For any compound, the therapeutically effective dose can be estimated initially either in cell culture assays, for example, P. falciparum strains, or in animal models, usually mice, rats or monkeys.

The animal model may also be used to determine the appropriate concentration range and route of administration. Such information can then be used to determine useful doses and routes for administration in humans.

The precise effective amount for a human subject will depend upon the severity of the disease state, general health of the subject, age, weight and gender of the subject, diet, time and frequency of administration, drug combination (s), reaction sensitivities and tolerance/response to therapy. This amount can be determined by routine experimentation and is within the judgement of the clinician. Generally, an effective dose will be from 0.01 mg/Kg to 100 mg/Kg, preferably 0.05 mg/Kg to 50 mg/Kg. Compositions may be administered individually to a patient or may be administered in combination with other agents, drugs.

### Pharmaceutical Compositions

In another aspect the invention provides novel pharmaceutical compositions comprising a therapeutically effective amount of the 4-amino-quinoline derivative of the invention.

The pharmaceutical composition of the invention comprising the 4-amino-quinoline derivative of the invention may further comprise known active principles such as artemisins (DHA, artesunate, etc.).

While a chemical compound of the invention for use in therapy may be administered in the form of the raw chemical compound, it is preferred to introduce the active ingredient, optionally in the form of a physiologically acceptable salt, or in the form of a prodrug, in a pharmaceutical composition together with one or more adjuvants, excipients, carriers, buffers, diluents and/or other customary pharmaceutical auxiliaries.

In a preferred embodiment, the invention provides pharmaceutical compositions comprising the 4-amino-quinoline derivative of the invention, or a pharmaceutically acceptable salt or derivative thereof, together with one or more pharmaceutically acceptable carriers, and, optionally, other therapeutic and/or prophylactic ingredients, know and used in the art. The carrier(s) must be “acceptable” in the sense of being compatible with the other ingredients of the formulation and not harmful to the recipient thereof.

Such carriers include antibodies and other polypeptides, genes and other therapeutic agents such as liposomes, provided that the carrier does not itself induce the production of antibodies harmful to the individual receiving the composition, and which may be administered without undue toxicity.

Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be prepared by any person skilled in the art, by use of standard methods and conventional techniques, appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/Kg i.v. and 100 mg/Kg p.o. Preferred ranges are from about 0.1 μg/kg to about 10 mg/Kg/day i.v., and from about 1 μg/kg to about 100 mg/Kg/day p.o.

### Methods of Therapy

In another aspect the invention provides a method of treatment, prevention or alleviation of an infectious disease, disorder or condition of a living animal body, including a human, which disorder, disease or condition is caused by a parasite of the genus Plasmodium, which method comprises the step of administering to such a living animal body in need thereof, a therapeutically effective amount of the 4-amino-quinoline derivative of the invention, or a pharmaceutically-acceptable addition salt thereof, or a prodrug thereof.

In the context of this invention the term “treatment” covers treatment, prevention, prophylaxis or alleviation, and the term “disease” covers illnesses, diseases, disorders and conditions related to the disease in question.

The preferred indications contemplated according to the invention are those stated above.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

### EXAMPLES

The invention is further illustrated with reference to the following examples, which are not intended to be in any way limiting to the scope of the invention as claimed.

### Example 1

### Synthesis of Derivatives 9a-s According to Scheme 1

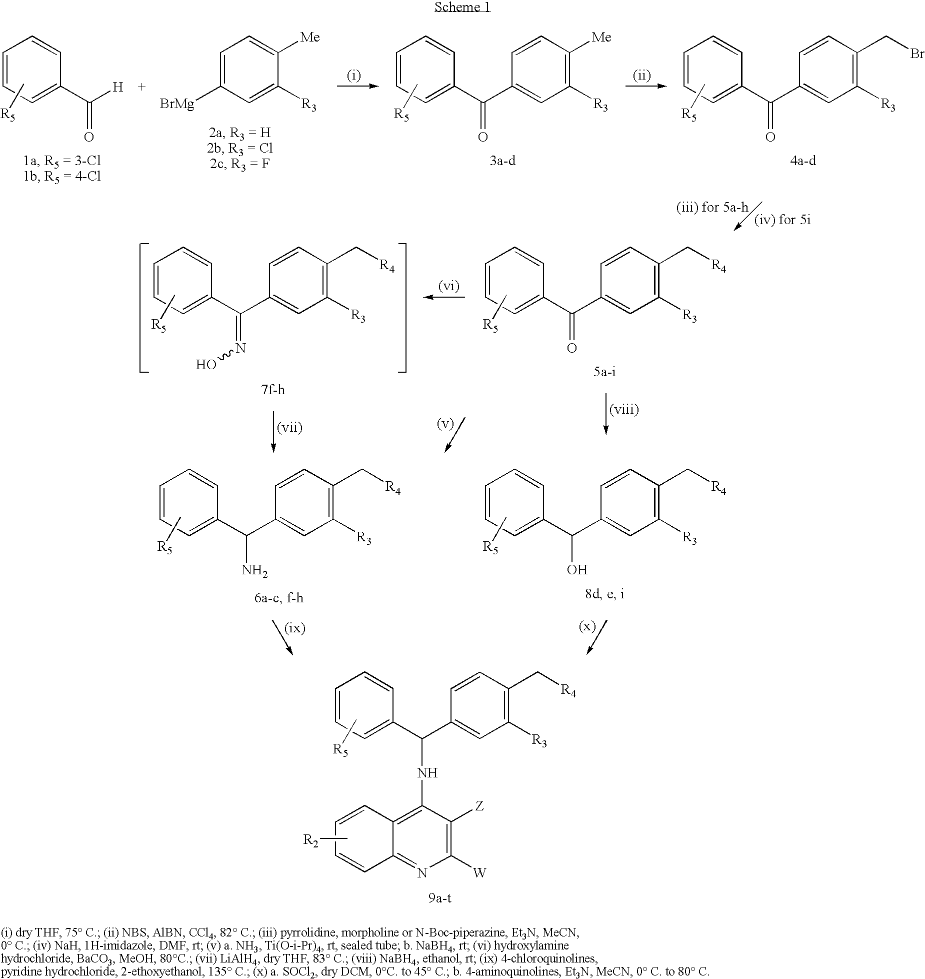

TABLE 1

| | | | | | |
|---|---|---|---|---|---|
| R₂ | R₃ | R₄ | R₅ | Z | W |

Compounds 3, 4

| | | | | | | |
|---|---|---|---|---|---|---|
| a | — | H | — | 3-Cl | — | — |
| b | — | H | — | 4-Cl | — | — |
| c | — | Cl | — | 3-Cl | — | — |
| d | — | F | — | 4-Cl | — | — |

Compounds 5-8

| | | | | | | |
|---|---|---|---|---|---|---|
| a | — | H | Pyrrolidin-1-yl | 3-Cl | — | — |
| b | — | H | Pyrrolidin-1-yl | 4-Cl | — | — |
| c | — | H | Morpholin-4-yl | 3-Cl | — | — |
| d | — | H | Morpholin-4-yl | 4-Cl | — | — |
| e | — | H | N-Boc-piperazin-2-yl | 4-Cl | — | — |
| f | — | Cl | Pyrrolidin-1-yl | 3-Cl | — | — |
| g | — | Cl | Morpholin-4-yl | 3-Cl | — | — |
| h | — | F | Pyrrolidin-1-yl | 4-Cl | — | — |
| i | — | H | 1H-Imidazol-1-yl | 4-Cl | — | — |

Compounds 9

| 9a | 7-Cl | H | Pyrrolidin-1-yl | 3-Cl | H | H |
|---|---|---|---|---|---|---|
| 9b | 7-Cl | H | Morpholin-4-yl | 3-Cl | H | H |
| 9c | 7-Cl | Cl | Pyrrolidin-1-yl | 3-Cl | H | H |
| 9d | 7-Cl | Cl | Morpholin-4-yl | 3-Cl | H | H |
| 9e | 7-Cl | H | Pyrrolidin-1-yl | 4-Cl | H | H |
| 9f | 7-Cl | H | Morpholin-4-yl | 4-Cl | H | H |
| 9g | 7-Cl | H | Piperazin-2-yl | 4-Cl | H | H |
| 9h | 7-Cl | F | Pyrrolidin-1-yl | 4-Cl | H | H |
| 9i | 7-CF₃ | Cl | Pyrrolidin-1-yl | 3-Cl | H | H |
| 9j | 7-CF₃ | Cl | Morpholin-4-yl | 3-Cl | H | H |
| 9k | 7-CF₃ | H | Pyrrolidin-1-yl | 4-Cl | H | H |
| 9l | 7-CF₃ | F | Pyrrolidin-1-yl | 4-Cl | H | H |
| 9m | 6-OMe | H | Pyrrolidin-1-yl | 3-Cl | H | H |
| 9n | 6-OMe | H | Morpholin-4-yl | 3-Cl | H | H |
| 9o | 6-OMe | Cl | Pyrrolidin-1-yl | 3-Cl | H | H |
| 9p | 6-OMe | H | Pyrrolidin-1-yl | 4-Cl | H | H |
| 9q | 6-OMe | H | Morpholin-4-yl | 4-Cl | H | H |
| 9r | 6-OMe | H | Piperazin-2-yl | 4-Cl | H | H |
| 9s | 6-OMe | H | Pyrrolidin-1-yl | 4-Cl | —CH═CH—C(Cl)═C— | |
| 9t | 7-Cl | H | 1H-Imidazol-1-yl | 4-Cl | H | H |

### (3-Chlorophenyl)(4-methylphenyl)methanone (3a)

To a stirred solution of magnesium turnings (1.13 g, 46.77 mmol) in anhydrous tetrahydrofuran (40 mL), a solution containing 4-bromotoluene 2 (8.0 g, 46.77 mmol) in dry THF (40 mL) was slowly added. The reaction mixture was stirred and heated under reflux until the reaction started, when the source of heat was removed until the exothermic reaction ceased. Heating was then continued under reflux for 1 h, when nearly all the magnesium had been consumed. Thereafter, the reaction was cooled to 0° C. and a solution of 3-chlorobenzaldehyde (1a, 10.0 mL, 93.54 mmol) in 70 mL of anhydrous tetrahydrofuran was added drop wise and the resulting solution was heated at 75° C. for 4 h. The reaction mixture was quenched with 50 mL of 20% of ammonium chloride solution. The aqueous layer was extracted with ethyl acetate (3×40 mL) and the combined organic layers were washed with brine, dried over Na₂SO₄ and evaporated under reduced pressure. The crude residue was purified by flash chromatography (2% ethyl acetate in hexane) to give 6a as a white solid (8.78 g, 70%); ¹H NMR (200 MHz, CDCl₃) δ 7.74-7.67 (m, 3H), 7.65-7.51 (m, 1H), 7.43-7.39 (m, 1H), 7.35-7.26 (m, 3H), 2.43 (s, 3H).

### (4-Chlorophenyl)(4-methylphenyl)methanone (3b)

Starting from 1b (5.0 g, 29.23 mmol) and 2, the title compound was prepared following the above described procedure and was obtained as a white solid (5.37 g, 80%); ¹H NMR (300 MHz, CDCl₃) δ 7.75-7.67 (m, 4H), 7.46-7.43 (m, 2H), 7.30-7.26 (m, 2H), 2.44 (s, 3H).

### (3-Chloro-4-methylphenyl)(3-chlorophenyl)methanone (3c)

Starting from 1a and 2b (15 g, 73 mmol), the title compound was prepared following the procedure described for 3a and after flash chromatography (ethyl acetate/n-hexane 40:1) was obtained as a white solid (18.0 g, 78%); ¹H NMR (CDCl₃) δ 7.78-7.75 (m, 2H), 7.64-7.55 (m, 3H), 7.45-7.44 (m, 2H), 2.46 (s, 3H).

### (4-Chlorophenyl)(3-fluoro-4-methylphenyl)methanone (3d)

Starting from 1b and 2c (10 g, 53 mmol), the title compound was prepared following the procedure described for 3a and after flash chromatography (ethyl acetate/n-hexane 1:30) was obtained as a white solid (11.2 g, 85%); ¹H NMR (300 MHz, CDCl₃) δ 7.71 (d, 2H, J=8.5 Hz), 7.47-7.44 (m, 4H), 7.32-7.26 (m, 1H), 2.36 (s, 3H); ESI MS m/z 250 (M+H)⁺.

### [4-(Bromomethyl)phenyl](3-chlorophenyl)methanone (4a)

To a solution of compound 3a (3.1 g, 13.5 mmol) in CCl₄ (40 mL) was added NBS (2.8 g, 15.6 mmol) and AIBN (catalytic amount). The solution was heated under reflux for 3 h. After cooling to room temperature, the succinimide was filtered off and the solvent was evaporated. The crude residue was purified by flash chromatography (5% ethyl acetate in hexane) to afford 4a as a white solid (3.4 g, 81%); ¹H NMR (200 MHz, CDCl₃) δ 7.74-7.63 (m, 4H), 7.59-7.38 (m, 4H), 4.49 (s, 2H).

### [4-(Bromomethyl)phenyl](4-chlorophenyl)methanone (4b)

Starting from 3b (3.6 g, 15.5 mmol), the title compound was prepared following the procedure described for 4a and was obtained as a white solid (3.9 g, 81%); 6 ¹H NMR (300 MHz, CDCl₃) δ 7.76-7.72 (m, 4H), 7.52-7.49 (m, 4H), 4.52 (s, 2H).

### [4-(Bromomethyl)-3-chlorophenyl](3-chlorophenyl)methanone (4c)

Starting from 3b (14 g, 53.7 mmol), the title compound was prepared following the procedure described for 4a and after flash chromatography (ethyl acetate/n-hexane 1:40) was obtained as a white solid (11.5 g, 62%); ¹H NMR (CDCl₃) δ 7.82-7.77 (m, 2H), 7.66-7.59 (m, 4H), 7.47-7.42 (m, 2H), 4.63 (s, 2H).

### [4-(bromomethyl)-3-fluorophenyl](4-chlorophenyl)methanone (4d)

Starting from 3d (5 g, 20.2 mmol), the title compound was prepared following the procedure described for 4a and after flash chromatography (ethyl acetate/n-hexane 1:30) was obtained as a white solid (5 g, 76%); ¹H NMR (300 MHz, CDCl₃) δ 7.75 (d, 2H, J=8.5 Hz), 7.54-7.47 (m, 5H), 4.55 (s, 2H); ESI MS m/z 329 (M+H)⁺.

### (3-Chlorophenyl)[4-(pyrrolidin-1-ylmethyl)phenyl]methanone (5a)

To a stirred solution of 4a (3.5 g, 11.30 mmol) in dry acetonitrile (75 mL) was added pyrrolidine (1.41 mL, 16.96 mmol) and triethylamine (3.14 mL, 22.61 mmol) at 0° C. and the resulting mixture was allowed to stir for 1 h at 0° C. Thereafter, the reaction was quenched with 2 mL of water and the solvent was evaporated under reduced pressure. The residue was treated with water and extracted with chloroform (2×40 mL). The combined organic layers were washed with brine, dried over sodium sulphate and evaporated. The residue was chromatographed (2% methanol in dichloromethane) to afford 5a as a yellow viscous oil (2.50 g, 73%); ¹H NMR (200 MHz, CDCl₃) δ 7.70-7.56 (m, 3H), 7.49-7.13 (m, 5H), 3.62 (s, 2H), 2.47 (m, 4H), 1.73 (m, 4H); ESI MS m/z 300 (M+H)⁺.

### (4-Chlorophenyl)[4-(pyrrolidin-1-ylmethyl)phenyl]methanone (5b)

Starting from 4b (3.0 g, 9.69 mmol), the title compound was prepared following the above described procedure and was obtained as a yellow viscous oil (2.16 g, 74%); ¹H NMR (300 MHz, CDCl₃) δ 7.76-7.68 (m, 4H), 7.48-7.39 (m, 4H), 3.68 (s, 2H), 2.52 (m, 4H), 1.77 (m, 4H); ESI MS m/z 300 (M+H)⁺.

### (3-Chlorophenyl)[4-(morpholin-4-ylmethyl)phenyl]methanone (5c)

Starting from 4a and morpholine, the title compound was prepared following the above described procedure and was obtained as a yellow viscous oil; ¹H NMR (300 MHz, CDCl₃) δ 7.76-7.73 (m, 3H), 7.67-7.63 (m, 1H), 7.56-7.53 (m, 1H), 7.48-7.44 (m, 2H), 7.41-7.38 (m, 1H), 3.74-3.71 (m, 4H), 3.57 (s, 2H), 2.49-2.47 (m, 4H).

### (4-Chlorophenyl)[4-(morpholin-4-ylmethyl)phenyl]methanone (5d)

Starting from 4b (0.58 g, 1.84 mmol), the title compound was prepared following the above described procedure and was obtained as a yellow oil (0.51 g, 87%); ¹H NMR (300 MHz, CDCl₃) δ 7.25-7.23 (m, 8H), 5.67 (s, 1H), 4.03 (bs, 1H), 3.57 (m, 4H), 3.41 (s, 2H), 3.38 (m, 4H); ESI MS m/z 318 (M+H)⁺.

### (4-Chlorophenyl){4-[4-(tert-butoxycarbonyl)piperazin-1-ylmethyl]phenyl}methanone (5e)

Starting from 4b (2.1 g, 5.1 mmol), the title compound was prepared following the above described procedure and was obtained as a white solid (1.86 g, 88%); ¹H NMR (300 MHz, CDCl₃) δ 7.29-7.20 (m, 6H), 5.69 (s, 1H), 3.98 (bs, 1H), 3.41 (s, 2H), 3.34 (m, 4H), 2.30 (m, 4H), 1.41 (s, 9H); ESI MS m/z 416 (M+H)⁺.

### [3-Chloro-4-(pyrrolidin-1-ylmethyl)phenyl](3-chlorophenyl)methanone (5f)

Starting from 4c (5.8 g, 17 mmol) and pyrrolidine, the title compound was prepared following the procedure described for 5a and after flash chromatography (CHCl₃/MeOH 20:1) was obtained as a white solid (5.1 g, 90%); ¹H NMR (CDCl₃) δ 7.78 (m, 2H), 7.65-7.47 (m, 4H), 7.44-7.42 (m, 1H), 3.82 (s, 2H), 2.63 (m, 4H), 1.84 (m, 4H); ESI MS m/z 334 (M+H)⁺.

### [3-Chloro-4-(morpholin-4-ylmethyl)phenyl](3-chlorophenyl)methanone (5g)

Starting from 4c (5.5 g, 16 mmol) and morpholine, the title compound was prepared following the procedure described for 5a and after flash chromatography (CHCl₃/MeOH 20:1) was obtained as a white solid (5.5 g, 98%); ¹H NMR (CDCl₃) δ 7.79-7.58 (m, 6H), 7.47-7.45 (m, 1H), 3.75 (m, 4H), 3.69 (s, 2H), 2.56 (m, 4H); ESI MS m/z 350 (M+H)⁺.

### (4-Chlorophenyl)[3-fluoro-4-(pyrrolidin-1-ylmethyl)phenyl]methanone (5h)

Starting from 4d (2.0 g, 6.1 mmol) and pyrrolidine, the title compound was prepared following the procedure described for 5a and after flash chromatography (CHCl₃/MeOH 9:1) was obtained as a white solid (1.7 g, 90%); ¹H NMR (300 MHz, CDCl₃) δ 7.71 (d, 2H, J=8.2 Hz), 7.55-7.40 (m, 4H), 7.26 (s, 1H), 3.72 (s, 2H), 2.55 (m, 4H), 1.77 (m, 4H); ESI MS m/z 318 (M+H)⁺.

### (4-Chlorophenyl)[4-(1H-imidazol-1-yl)methylphenyl]methanone (5i)

To a suspension of 310 mg (12.9 mmol) of NaH (as 60% oil dispersion) in 30 mL of dry DMF, imidazole (881 mg, 12.9 mmol) was added and the resulting mixture was stirred for 1 h at rt. Thereafter, the mixture was cooled to 0° C. and 4b (2.0 g, 6.5 mmol) was added, the solution was stirred at rt for 1 h and the solvent was removed. H₂O (20 mL) was added to the solid residue, the aqueous phase was extracted with chloroform (40 mL×3), the combined organic layers were dried over Na₂SO₄, filtered, and the solvent evaporated. The crude residue was purified by flash chromatography (EtOAc/n-Hexane 1:10) to afford the title compound as a white solid (1.4 g, 75%). ¹H-NMR (CDCl₃) δ 7.75-7.68 (m, 4H), 7.57 (m, 1H), 7.43 (d, 2H, J=8.5 Hz), 7.23 (d, 2H, J=8.2 Hz), 7.11 (m, 1H), 6.92 (m, 1H), 5.2 (s, 2H). ESI-MS m/z 297 (M+H)⁺.

### [3-Chloro-4-(pyrrolidin-1-ylmethyl)phenyl](3-chlorophenyl)methanone oxime (7f)

To a solution of 5f (3.3 g, 10.1 mmol) in methanol (150 mL), hydroxylamine hydrochloride (1.6 g, 23.3 mmol) and BaCO₃ (4.6 g, 23.3 mmol) were added and the mixture was heated under reflux for 18 h. Thereafter, the white precipitate was removed by filtration and the solvent was evaporated under reduced pressure. After evaporation of the solvent, the crude oxime was obtained as colourless oil and used without further purification; ESI MS m/z 349 (M+H)⁺.

### [3-chloro-4-(morpholin-4-ylmethyl)phenyl](3-chlorophenyl)methanone oxime (7g)

The title compound was prepared following the above described procedure starting from 5g (2.9 g, 8.3 mmol). The crude product was used in the next step without further purification; ESI MS m/z 365 (M+H)⁺.

### (4-Chlorophenyl)[3-fluoro-4-(pyrrolidin-1-ylmethyl)phenyl]methanone oxime (7h)

The title compound was prepared following the above described procedure starting from 5h (2.5 g, 12.6 mmol). The crude product was used in the next step without further purification; ESI MS m/z 347 (M+H)⁺.

### (±)-1-(3-Chlorophenyl)-1-[4-(pyrrolidin-1-ylmethyl)phenyl]methylamine (6a)

To a mixture of 5a (2.8 g, 9.4 mmol) in a 2.0 M solution of ammonia in EtOH (24 mL, 50 mmol), titanium(IV) isopropoxide (5.5 mL, 18.8 mmol) was added and the reaction mixture was allowed to stir for 16 h. Subsequently, sodium borohydride (0.53 g, 14.0 mmol) was added and the mixture was stirred at rt for 3 h and then poured into a 2.0 M solution of ammonium hydroxide. The inorganic precipitate was filtered off and the aqueous phase was extracted with CHCl₃. The organic extracts were washed with brine, dried over Na₂SO₄ and the solvent was removed. The residue was purified by flash-chromatography eluting 5% MeOH in CHCl₃ to afford 0.83 g (30%) of 7a as colourless oil; ¹H NMR (200 MHz, CDCl₃) δ 7.40-7.36 (m, 1H), 7.28-7.21 (m, 4H), 7.21-7.17 (m, 3H), 5.14 (s, 1H), 3.61 (s, 2H), 2.55-2.53 (m, 4H), 2.28 (bs, 2H), 1.88-1.85 (m, 4H); ESI MS m/z 301 (M+H)⁺.

### (±)-1-(4-Chlorophenyl)-1-[4-(pyrrolidin-1-ylmethyl)phenyl]methylamine (6b)

Starting from 5b, the title compound was prepared following the procedure described for compound 6a and was obtained as a yellow viscous oil; ¹H NMR (300 MHz, CDCl₃) δ 7.34-7.21 (m, 8H), 5.17 (s, 1H), 3.58 (s, 2H), 2.52-2.46 (m, 4H), 2.0 (bs, 2H), 1.90-1.72 (m, 4H); ESI MS m/z 301 (M+H)⁺.

### (±)-1-(3-Chlorophenyl)-1-[4-(morpholin-4-ylmethyl)phenyl]methylamine (6c)

Starting from 5c, the title compound was prepared following the procedure described for compound 6a and was obtained as a yellow viscous oil; ¹H NMR (300 MHz, CDCl₃) δ 7.35-7.11 (m, 8H), 5.07 (s, 1H), 3.62-3.59 (m, 4H), 3.39 (s, 2H), 2.36-2.33 (m, 4H), 2.04 (bs, 2H). ESI MS m/z 317 (M+H)⁺.

### (±)-1-(3-Chlorophenyl)-1-[3-chloro-4-(pyrrolidin-1-ylmethyl)phenyl]methylamine (6f)

To suspension of LiAlH₄ (1.0 g, 28.0 mmol) in dry THF (30 mL), heated under reflux, a solution of 7f (3.2 g, 9.35 mmol) in THF (15 mL) was added dropwise. The resulting mixture was heated under reflux for 1 h, then cooled to 0° C. and quenched with ethanol and H₂O. Thereafter, the solvent was evaporated and the residue was suspended in 20 mL of H₂O and the aqueous layer was extracted with CHCl₃ (3×40 ml). The combined organic layers were dried over Na₂SO₄ and evaporated under reduced pressure. The crude residue was purified by flash chromatography (CHCl₃/MeOH 20:1) to give the title compound as a yellow oil (1.5 g, 50%); ¹H NMR (CDCl₃) δ 7.44-7.24 (m, 3H), 7.23-7.19 (m, 4H), 5.13 (s, 1H), 3.72 (s, 2H), 2.57 (m, 4H), 1.79 (m, 6H); ESI MS m/z 335 (M+H)⁺.

### (±)-1-(3-Chlorophenyl)-1-[3-chloro-4-(morpholin-4-ylmethyl)phenyl]methylamine (6g)

The title compound was prepared from 7g (2.9 g, 8.0 mmol) following the procedure described for 6f and was obtained as a yellow oil (1.7 g, 62%). ¹H NMR (CDCl₃) δ 7.43-7.38 (m, 3H), 7.26-7.20 (m, 4H), 5.12 (s, 1H), 3.71-3.68 (m, 4H), 3.57 (s, 2H), 2.51-2.48 (m, 4H), 1.92 (bs, 2H); ESI MS m/z 351 (M+H)⁺.

### (±)-1-(4-Chlorophenyl)-1-[3-fluoro-4-(pyrrolidin-1-ylmethyl)phenyl]methylamine (6h)

The title compound was prepared from 7h (2.0 g, 5.99 mmol) following the procedure described for 6f and was obtained as a yellow oil (1.62 g, 85%). ¹H NMR (300 MHz, CDCl₃) δ 7.44-7.24 (m, 3H), 7.23-7.08 (m, 4H), 5.11 (s, 1H), 3.69 (s, 2H), 2.69 (m, 4H), 2.94 (m, 6H); ESI MS m/z 320 (M+H)⁺.

### (±)-(4-Chlorophenyl)[4-(morpholinomethyl)phenyl]methanol (8d)

To a stirred solution of 5d (0.58 g, 1.84 mmol) in ethanol (10 mL), sodium borohydride (90.0 mg, 2.5 mmol) was added at 0° C. and the resulting mixture was allowed to stir for 1 h at 0° C. Thereafter, the reaction mixture was quenched by dropwise addition of water (5 mL) and the solvent was evaporated under reduced pressure. The residue was treated with water and extracted with chloroform (3×20 mL). The combined organic layers were washed with brine, dried over Na₂SO₄ and evaporated. The residue was chromatographed (6% methanol in chloroform) to afford 8d as a brown oil (0.51 g, 87%); ¹H NMR (300 MHz, CDCl₃) δ 7.25-7.23 (m, 8H), 5.67 (s, 1H), 4.03 (bs, 1H), 3.57 (m, 4H), 3.41 (s, 2H), 3.38 (m, 4H); ESI MS m/z 318 (M+H)⁺.

### (±)-(4-Chlorophenyl)[4-(1-tert-butoxycarbonylpiperazin-4-ylmethyl)phenyl]methanol (8e)

Starting from 5e (2.1 g, 5.1 mmol), the title compound was prepared following the procedure described for 8d and was obtained as a white solid (1.86 g, 88%); ¹H NMR (300 MHz, CDCl₃) δ 7.29-7.20 (m, 8H), 5.69 (s, 1H), 3.98 (bs, 1H), 3.41 (s, 2H), 3.34 (m, 4H), 2.30 (m, 4H), 1.41 (s, 9H); ESI MS m/z 417 (M+H)⁺.

### (±)-(4-chlorophenyl)[4-(1H-imidazol-1-yl)methylphenyl]methanol (8i)

Starting from 5i (831 mg, 2.8 mmol), the title compound was prepared following the procedure described for 8d and was obtained as a white solid (795 mg, 95%); ¹H-NMR (CDCl₃) δ 7.34-7.26 (m, 7H), 7.05-7.02 (m, 2H), 6.88 (m, 1H), 6.81 (m, 1H), 5.79 (s, 1H), 5.04 (s, 2H); ESI-MS m/z 299 (M+H)⁺.

### (±)-7-Chloro-N-{(3-chlorophenyl)[4-(pyrrolidin-1-ylmethyl)phenyl]methyl}-4-aminoquinoline (9a)

To a solution of 7a (0.83 g, 2.8 mmol) in 2-ethoxyethanol (10 mL), 4,7-dichloroquinoline (0.57 g, 2.9 mmol) and pyridium chloride (0.35 g, 3.0 mmol) were added. The resulting mixture was heated to 135° C. for 5 h and then was cooled to rt. Triethylamine (0.5 mL) was added and the solvent was evaporated under reduced pressure. The residue was purified by flash-chromatography (5% MeOH in CHCl₃) to afford 7a as a white low-melting solid (0.9 g, 70%). ¹H NMR (300 MHz, CDCl₃) δ 8.44 (d, 1H, J=5.3 Hz), 7.98 (d, 1H, J=2.0 Hz), 7.72 (d, 1H, J=8.8 Hz), 7.41-7.26 (m, 9H), 6.23 (d, 1H, J=5.3 Hz), 5.68 (d, 1H, J=4.4 Hz), 5.39 (d, 1H, J=4.4 Hz), 3.68 (s, 2H), 2.61-2.59 (m, 4H), 1.85-1.81 (m, 4H).

### (±)-7-Chloro-N-{(3-chlorophenyl)[4-(morpholin-4-ylmethyl)phenyl]methyl}-4-aminoquinoline (9b)

Starting from 7c and 4,7-dichloroquinoline, the title compound was prepared following the procedure described for 9a; ¹H NMR (300 MHz, CDCl₃) δ 8.45 (d, 1H, J=5.3 Hz), 8.0 (s, 1H), 7.72 (d, 1H, J=9.1 Hz), 7.41-7.26 (m, 9H), 6.25 (d, 1H, J=5.6 Hz), 5.70 (d, 1H, J=4.1 Hz), 5.41 (d, 1H, J=3.2 Hz), 3.72-3.70 (m, 4H), 3.50 (s, 2H), 2.47-2.45 (m, 4H).

### (±)-7-Chloro-N-{[3-chloro-4-(pyrrolidin-1-ylmethyl)phenyl](3-chlorophenyl)methyl}-4-aminoquinoline (9c)

Starting from 7f (156 mg, 0.467 mmol) and 4,7-dichloroquinoline, the title compound was prepared following the procedure described for 9a and was obtained as a white solid (120 mg, 52%); ¹H NMR (300 MHz, CDCl₃) δ 8.45 (d, 1H, J=5.3 Hz), 7.99 (s, 1H), 7.73 (d, 1H, J=8.8 Hz), 7.52 (d, 1H, J=7.9 Hz), 7.40 (d, 1H, J=8.2 Hz), 7.30-7.20 (m, 6H), 6.22 (d, 1H, J=5.3 Hz), 5.65 (d, 1H, J=4.1 Hz), 5.37 (d, 1H, J=4.1 Hz), 3.74 (s, 2H), 2.60 (m, 4H), 1.81 (m, 4H); ESI MS m/z 496 (M+H)⁺.

### (±)-7-Chloro-N-{[3-chloro-4-(morpholin-4-ylmethyl)phenyl](3-chlorophenyl)methyl}-4-aminoquinoline (9d)

Starting from 7g (260 mg, 0.74 mmol) and 4,7-dichloroquinoline, the title compound was prepared following the procedure described for 9a and was obtained as a white solid (220 mg, 58%); ¹H NMR (CDCl₃) δ 8.35 (d, 1H, J=5.3 Hz), 7.88-7.83 (m, 2H), 7.46 (d, 1H, J=7.9 Hz), 7.30-7.19 (m, 7H), 6.19 (d, 1H, J=5.3 Hz), 5.84 (d, 1H, J=4.1 Hz), 5.65 (d, 1H, J=4.1 Hz), 3.66 (m, 4H), 3.55 (s, 2H), 2.47 (m, 4H); ESI MS m/z 512 (M+H)⁺.

### (±)-7-Chloro-N-{(4-chlorophenyl)[4-(pyrrolidin-1-ylmethyl)phenyl]methyl}-4-aminoquinoline (9e)

Starting from 7b and 4,7-dichloroquinoline, the title compound was prepared following the procedure described for 9a; ¹H NMR (300 MHz, CDCl₃) δ 8.42 (d, 1H, J=5.3 Hz), 7.97 (d, 1H, J=2.0 Hz), 7.72 (d, 1H, J=8.8 Hz), 7.40-7.26 (m, 9H), 6.23 (d, 1H, J=5.3 Hz), 5.71 (d, 1H, J=4.1 Hz), 5.41 (d, 1H, J=4.1 Hz), 3.63 (s, 2H), 2.55-2.54 (m, 4H), 1.82-1.78 (m, 4H).

### (±)-7-Chloro-N-{(4-chlorophenyl)[4-(morpholin-4-ylmethyl)phenyl]methyl}-4-aminoquinoline (9f)

To a solution of 8d (0.25 g, 0.78 mmol) in dry DCM (8 mL) was added a solution of SOCl₂ (451 μL, 6.18 mmol) in dry DCM (4 mL) at 0° C., and the mixture was stirred at 0° C. for 20 min and thereafter heated to 45° C. for 4 h. The volatiles were removed and the residue was treated with dry MeCN (3×4 mL) and concentrated under reduced pressure in order to remove residual SOCl₂. The resulting hydrochloride salt was suspended in dry MeCN (12 mL) and to this solution was added a solution containing triethylamine (430 μL, 3.0 mmol) and 4-amino-7-chloroquinoline (0.16 g, 0.94 mmol) at 0° C. Thereafter, the reaction mixture was heated to 80° C. for 6 h. The solvent was evaporated under reduced pressure and the residue was treated with water and extracted with ethyl acetate (4×30 mL). The combined organic layers were dried over Na₂SO₄ and concentrated in vacuo. The crude residue was purified by flash column chromatography (2% MeOH in CHCl₃) to afford 9f as a light yellow solid (0.11 g, 32%); ¹H NMR (300 MHz, CDCl₃) δ 8.43 (d, 1H, J=5.2 Hz), 7.98 (s, 1H), 7.71 (d, 1H, J=9.0 Hz), 7.39-7.26 (m, 9H), 6.23 (d, 1H, J=5.2 Hz), 5.70 (d, 1H, J=4.1 Hz), 5.38 (d, 1H, J=3.8 Hz), 3.70 (m, 4H), 3.49 (s, 2H), 2.44 (m, 4H); ESI MS m/z 478 (M+H)⁺.

### (±)-7-Chloro-N-{(4-chlorophenyl)[4-(piperazin-1-ylmethyl)phenyl]methyl}-4-aminoquinoline (9g)

Starting from 8e (0.12 g, 0.28 mmol) and 4,7-dichloroquinoline, the title compound was prepared following the procedure described for 9f and was obtained as a white solid (90.0 mg, 68%); ¹H NMR (300 MHz, CDCl₃) δ 8.40 (d, 1H, J=5.2 Hz), 7.95 (d, 1H, J=2.0 Hz), 7.72 (d, 1H, J=8.7 Hz), 7.36-7.23 (m, 9H), 6.21 (d, 1H, J=5.2 Hz), 5.68 (d, 1H, J=4.3 Hz), 5.46 (d, 1H, J=4.3 Hz), 3.47 (s, 2H), 2.86 (m, 4H), 2.40 (m, 4H), 1.84 (s, 1H); ESI MS m/z 477 (M+H)⁺.

### (±)-7-Chloro-N-{(4-chlorophenyl)[3-fluoro-4-(pyrrolidin-1-ylmethyl)phenyl]methyl}-4-aminoquinoline (9h)

Starting from 7h (100 mg, 0.32 mmol) and 4,7-dichloroquinoline, the title compound was prepared following the procedure described for 9a and was obtained as a white solid (120 mg, 78%); ¹H NMR (300 MHz, CDCl₃) δ 8.41 (d, 1H, J=4.7 Hz), 7.95 (s, 1H), 7.75 (d, 1H, J=8.8 Hz), 7.43-7.28 (m, 6H) 7.09 (d, 1H, J=7.6 Hz), 6.98 (d, 1H, J=10.3 Hz), 6.21 (d, 1H, J=4.7 Hz), 5.67 (s, 1H), 5.51 (s, 1H), 3.65 (s, 2H), 2.54 (m, 4H), 1.77 (m, 4H); ESI MS m/z 480 (M+H)⁺.

### (±)-N-{[3-Chloro-4-(pyrrolidin-1-ylmethyl)phenyl](3-chlorophenyl)methyl}-7-trifluoromethyl-4-aminoquinoline (9i)

Starting from 7f (573 mg, 1.71 mmol) and 7-trifluoromethyl-4-chloroquinoline, the title compound was prepared following the procedure described for 9a and was obtained as a white solid (420 mg, 46%). ¹H NMR (CDCl₃) δ 8.48 (d, 1H, J=5.3 Hz), 8.25 (s, 1H), 8.01 (d, 1H, J=8.8 Hz), 7.57 (d, 1H, J=8.8 Hz), 7.49 (d, 1H, J=7.9 Hz) 7.31-7.20 (m, 6H), 6.31 (d, 1H, J=5.3 Hz), 5.75 (d, 1H, J=4.1 Hz), 5.68 (d, 1H, J=4.1 Hz), 3.71 (s, 2H), 2.57 (m, 4H), 1.78 (m, 4H); ESI MS m/z 530 (M+H)⁺.

### (±)-N-{[3-Chloro-4-(morpholin-4-ylmethyl)phenyl](3-chlorophenyl)methyl}-7-trifluoromethyl-4-aminoquinoline (9j)

Starting from 7g (293 mg, 0.83 mmol) and 7-trifluoromethyl-4-chloroquinoline, the title compound was prepared following the procedure described for 9a and was obtained as a white solid (250 mg, 55%). ¹H NMR (CDCl₃) δ 8.56 (d, 1H, J=5.3 Hz), 8.31 (s, 1H), 7.91 (d, 1H, J=8.8 Hz), 7.63 (d, 1H, J=9.1 Hz), 7.52 (d, 1H, J=8.2 Hz), 7.35-7.22 (m, 6H), 6.32 (d, 1H, J=5.3 Hz), 5.67 (d, 1H, J=4.1 Hz), 5.40 (d, 1H, J=4.1 Hz), 3.74-3.71 (m, 4H), 3.61 (s, 2H), 2.54-2.51 (m, 4H); ESI MS m/z 546 (M+H)⁺.

### (±)-N-{(4-Chlorophenyl)[4-(pyrrolidin-1-ylmethyl)phenyl]methyl}-7-trifluoromethyl-4-aminoquinoline (9k)

Starting from 7b and 7-trifluoro-4-chloroquinoline, the title compound was prepared following procedure described for 9a; ¹H NMR (300 MHz, CDCl₃) δ 8.52 (d, 1H, J=5.3 Hz), 8.29 (s, 1H), 7.91 (d, 1H, J=8.8 Hz), 7.61 (dd, 1H, J=1.8, 8.8 Hz), 7.39-7.28 (m, 9H), 6.33 (d, 1H, J=4.4 Hz), 5.48 (d, 1H, J=4.4 Hz), 3.65 (s, 2H), 2.57-2.55 (m, 4H), 1.84-1.80 (m, 4H).

### (±)-N-{(4-Chlorophenyl)[3-fluoro-4-(pyrrolidin-1-ylmethyl)phenyl]methyl}-7-trifluoromethyl-4-aminoquinoline (9l)

Starting from 7h (196 mg, 0.62 mmol) and 7-trifluoromethyl-4-chloroquinoline, the title compound was prepared following the procedure described for 9a and was obtained as a white solid (98 mg, 35%); ¹H NMR (300 MHz, CDCl₃) δ 8.53 (d, 1H, J=5.0 Hz), 8.30 (s, 1H), 7.91 (d, 1H, J=7.4 Hz), 7.62 (d, 1H, J=8.8 Hz) 7.43-7.26 (m, 5H), 7.11 (d, 1H, J=7.9 Hz), 7.0 (d, 1H, J=10.3 Hz), 6.32 (d, 1H, J=5.0 Hz), 5.71 (d, 1H, J=4.1), 5.46 (d, 1H, J=3.8 Hz), 3.67 (s, 2H), 2.56 (m, 4H), 1.79 (m, 4H); ESI MS m/z 514 (M+H)⁺.

### (±)-6-Methoxy-N-{(3-chlorophenyl)[4-(pyrrolidin-1-ylmethyl)phenyl]methyl}-4-aminoquinoline (9m)

Starting from 7a (178 mg, 0.56 mmol) and 6-methoxy-4-chloroquinoline, the title compound was prepared following the procedure described for 9a and was obtained as a white solid (140 mg, 48%); ¹H NMR (300 MHz, CDCl₃) δ 8.28 (d, 1H, J=5.3 Hz), 7.91 (d, 1H, J=9.4 Hz), 7.36-7.25 (m, 9H), 7.08 (s, 1H), 6.22 (d, 1H, J=5.3 Hz), 5.70 (d, 1H, J=4.1 Hz), 5.47 (d, 1H, J=3.8 Hz), 3.85 (s, 3H), 3.62 (s, 2H), 2.55 (m, 4H), 1.79 (m, 4H); ¹³C NMR (300 MHz, CDCl₃) δ 157.3, 148.4, 148.0, 143.9, 143.4, 139.7, 139.4, 135.0, 131.4, 130.5, 130.0, 128.2, 127.9, 127.7, 125.9, 120.8, 119.6, 101.5, 99.7, 62.0, 60.4, 56.0, 54.5, 23.7; ESI MS m/z 458 (M+H)⁺.

### (±)-N-{(3-Chlorophenyl)[4-(morpholin-4-ylmethyl)phenyl]methyl}-6-methoxy-4-aminoquinoline (9n)

Starting from 7c (910 mg, 2.87 mmol) and 6-methoxy-4-chloroquinoline, the title compound was prepared following the procedure described for 9a and was obtained as a white solid (550 mg, 40%); ¹H NMR (CDCl₃) δ 8.34 (d, 1H, J=5.0 Hz), 7.94 (d, 1H, J=9.4 Hz), 7.36-7.26 (m, 9H), 7.02 (s, 1H), 6.23 (d, 1H, J=5.0 Hz), 5.69 (d, 1H, J=3.8 Hz), 5.24 (d, 1H, J=3.8 Hz), 3.84 (s, 3H), 3.69 (m, 4H), 3.47 (s, 2H), 2.43 (m, 4H); ¹³C NMR (CDCl₃) δ 157.3, 148.7, 147.6, 144.0, 143.5, 139.9, 138.4, 135.2, 131.8, 130.6, 130.2, 128.3, 127.7, 127.7, 125.7, 120.3, 119.7, 101.6, 99.7, 67.2, 63.2, 62.0, 56.0, 53.9; ESI MS m/z 474 (M+H)⁺.

### (±)-N-{[3-Chloro-4-(pyrrolidin-1-ylmethyl)phenyl](3-chlorophenyl)methyl}-6-methoxy-4-aminoquinoline (9o)

Starting from 7f (910 mg, 2.87 mmol) and 6-methoxy-4-chloroquinoline, the title compound was prepared following the procedure described for 9a and was obtained as a white solid (250 mg, 30%); ¹H NMR (CDCl₃) δ 8.37 (d, 1H, J=5.3 Hz), 7.95 (d, 1H, J=9.4 Hz), 7.51 (d, 1H, J=7.9 Hz), 7.36-7.22 (m, 7H), 6.98 (d, 1H, J=2.0 Hz), 6.23 (d, 1H, J=5.3 Hz), 5.66 (d, 1H, J=3.8 Hz), 5.09 (d, 1H, J=3.8 Hz), 3.93 (s, 3H), 3.74 (s, 2H), 2.61 (m, 4H), 1.81 (m, 4H); ESI MS m/z 492 (M+H)⁺.

### (±)-6-Methoxy-N-{(4-chlorophenyl)[4-(pyrrolidin-1-ylmethyl)phenyl]methyl}-4-aminoquinoline (9p)

Starting from 7b and 6-methoxy-4-chloroquinoline, the title compound was prepared following the procedure described for 9a; ¹H NMR (300 MHz, CDCl₃) δ 8.31 (d, 1H, J=5.0 Hz), 7.93 (d, 1H, J=9.1 Hz), 7.38-7.26 (m, 9H), 7.03 (d, 1H, J=2.6 Hz, 1H), 6.22 (d, 1H, J=5.3 Hz), 5.71 (d, 1H, J=4.1 Hz), 5.32 (d, 1H, J=4.1 Hz), 3.88 (s, 3H), 3.65 (s, 2H), 2.57-2.55 (m, 4H), 1.84-1.79 (m, 4H).

### (±)-N-{(4-Chlorophenyl)[4-(morpholin-4-ylmethyl)phenyl]methyl}-6-methoxy-4-aminoquinoline (9q)

Starting from 8d (0.25 g, 0.78 mmol) and 6-methoxy-4-aminoquinoline, the title compound was prepared following the procedure described for 9f and was obtained as brown oil (52 mg, 14%); ¹H NMR (300 MHz, CDCl₃) δ 8.32 (d, 1H, J=5.2 Hz), 7.91 (d, 1H, J=9.3 Hz), 7.34-7.26 (m, 9H), 7.03 (s, 1H), 6.23 (d, 1H, J=5.2 Hz), 5.71 (d, 1H, J=3.8 Hz), 5.28 (d, 1H, J=3.5 Hz), 3.85 (s, 3H), 3.70 (m, 4H), 3.49 (s, 2H), 2.44 (m, 4H); ESI MS m/z 474 (M+H)⁺.

### (±)-N-{(4-Chlorophenyl)[4-(piperazin-1-ylmethyl)phenyl]methyl}-6-methoxy-4-aminoquinoline (9r)

Starting from 8e (80 mg, 0.14 mmol) and 6-methoxy-4-aminoquinoline, the title compound was prepared following the procedure described for 9f and was obtained as a light yellow oil (50 mg, 75.75%); ¹H NMR (300 MHz, CDCl₃) δ 8.31 (d, 1H, J=4.9 Hz), 7.90 (d, 1H, J=9.0 Hz), 7.32-7.25 (m, 9H), 7.03 (s, 1H), 6.21 (d, 1H, J=4.9 Hz), 5.69 (d, 1H, J=4.1 Hz), 5.27 (s, 1H), 3.87 (s, 3H), 3.47 (s, 2H), 2.87 (m, 4H), 2.40 (m, 4H), 1.90 (s, 1H); ESI MS m/z 473 (M+H)⁺.

### (±)-6-Chloro-N-{(4-chlorophenyl)[4-(pyrrolidin-1-ylmethyl)phenyl]methyl}-2-methoxy-9-aminoacridine (9s)

Starting from 7b and 6,9-dichloro-2-methoxyacridine, the title compound was prepared following the procedure described for 9a; ¹H NMR (300 MHz, CDCl₃) δ 8.07 (d, 1H, J=2.0 Hz), 7.97 (d, 1H, J=9.7 Hz), 7.48 (d, 1H, J=9.4 Hz), 7.35-7.29 (m, 7H), 7.20 (d, 2H, J=7.9 Hz), 7.14 (dd, 1H, J=2.0, 9.4 Hz), 6.84 (d, 1H, J=2.6 Hz), 5.87 (d, 1H, J=8.5 Hz), 4.80 (d, 1H, J=8.8 Hz), 3.60 (s, 2H), 3.42 (s, 3H), 2.51-2.46 (m, 4H), 1.81-1.77 (m, 4H).

### (±)-7-Chloro-N-{[4-(1H-imidazol-1-yl)methylphenyl](4-chlorophenyl)methyl}-4-aminoquinoline (9t)

Starting from 8i (795 mg, 2.67 mmol) and 7-chloro-4-aminoquinoline, the title compound was prepared following the procedure described for 9f and was obtained as a white solid (550 mg, 45%); ¹H-NMR (CDCl₃) δ 8.34 (d, 1H, J=5.3 Hz), 7.89 (d, 1H, J=2.1 Hz), 7.85 (d, 1H, J=9.1 Hz), 7.39 (s, 1H), 7.34-7.19 (m, 8H), 7.09 (d, 1H, J=7.9 Hz), 6.98 (m, 1H), 6.85 (m, 1H), 6.17 (d, 1H, J=5.3 Hz), 5.94 (d, 1H, J=4.7 Hz), 5.70 (d, 1H, J=4.7 Hz), 5.05 (s, 2H); ESI-MS m/z 459 (M+H)⁺.

### Example 2

### Synthesis of Derivatives 15a-d According to Scheme 2

SCHEME 2

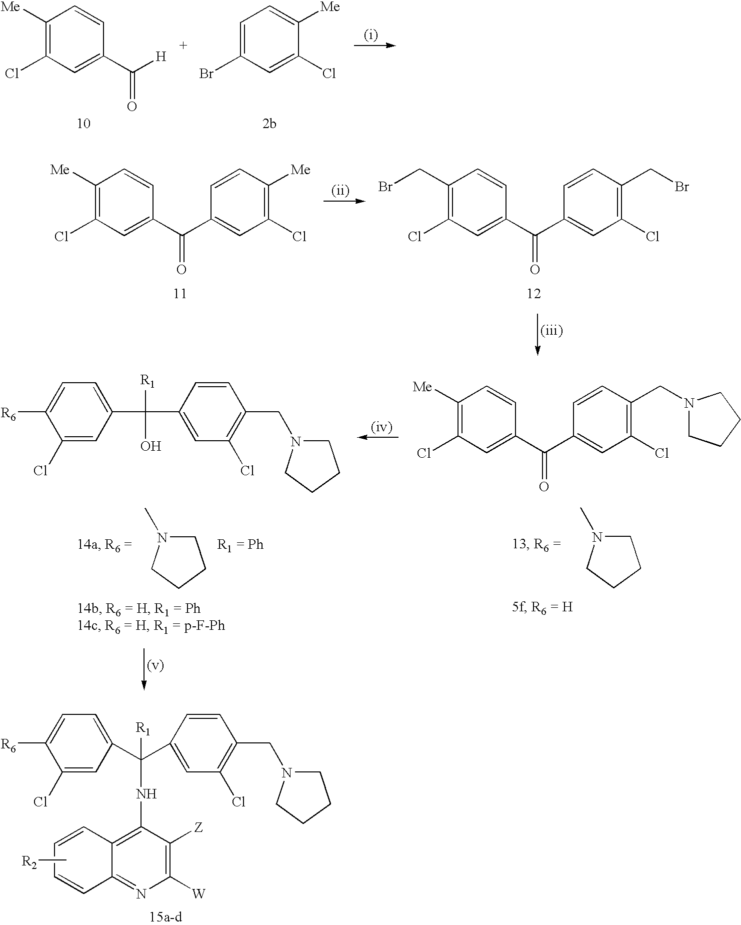

| | R₁ | R₂ | R₆ | Z | W |
|---|---|---|---|---|---|
| 15a | Ph | 7-Cl | H | H | H |
| 15b | p-F-Ph | 7-Cl | H | H | |

| | | | | |
|---|---|---|---|---|
| 15c | Ph | 6-OMe | H | —CH═CH—C(Cl)═C— |

| | | | | | |
|---|---|---|---|---|---|
| 15d | Ph | 7-Cl | 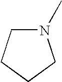 | H | H |

Scheme 6
(i) Mg, dry THF, 75° C., 4 h;
(ii) NBS, AlBN, CCl₄, 82° C., 4 h;
(iii) pyrrolidine, Et₃N, MeCN, 0° C., 1 h;
(iv) bromobenzene or 1-bromo-4-fluorobenzene, Mg, dry THF, 75° C. 8 h;
(v) (a) SOCl₂, dry DCM, 0° C. to 45° C., 4 h, (b) 4-aminooquinolines, Et₃N, MeCN, 0° C. to 80° C. 4 h

### bis(3-Chloro-4-methylphenyl)methanone (11)

Starting from 2b (2.0 g, 9.73 mmol) and 10 (3.0 g, 19.46 mmol), the title compound was prepared using magnesium turnings (0.24 g, 9.73 mmol) as described for 3a, and was obtained as a white solid (2.5 g, 92%); ¹H NMR (300 MHz, CDCl₃) δ 7.75 (d, 2H, J=1.4 Hz), 7.56-7.52 (m, 2H), 7.34-7.31 (m, 2H), 2.44 (s, 6H).

### bis(4-Bromomethyl-3-chlorophenyl)methanone (12)

To a solution of 11 (2.7 g, 9.67 mmol) in CCl₄ (30 mL), NBS (3.78 g, 21.27 mmol) and AIBN (catalytic amount) were added. The solution was heated under reflux for 4 h. After cooling to rt, the succinimide was filtered off and the solvent was evaporated. The residue was purified by flash chromatography (2% ethyl acetate in hexane) to afford 12 as a white solid (1.8 g, 42.65%); ¹H NMR (300 MHz, CDCl₃) δ 7.79 (m, 2H), 7.64-7.55 (m, 4H), 4.61 (s, 4H).

### bis[3-Chloro-4-(pyrrolidin-1-ylmethyl)phenyl]methanone (13)

To a stirred solution of 12 (1.8 g, 4.12 mmol) in dry MeCN (20 mL), cooled to 0° C., pyrrolidine (0.83 mL, 9.88 mmol) and triethylamine (2.3 mL, 16.45 mmol) were added and the resulting mixture was allowed to stir for 1 h at 0° C. Thereafter, the reaction mixture was quenched with 2 mL of water and the solvent was evaporated under reduced pressure. The residue was treated with water and extracted with DCM (3×30 mL). The combined organic layers were washed with brine, dried over Na₂SO₄ and evaporated. The residue was chromatographed (2% methanol in chloroform) to afford 13 as a yellow viscous oil (1.5 g, 88%); ¹H NMR (400 MHz, CDCl₃) δ 7.77 (m, 2H), 7.60-7.56 (m, 4H), 3.97 (s, 4H), 2.57 (m, 8H), 1.76 (m, 8H); ESI MS m/z 417 (M+H)⁺.

### bis[3-Chloro-4-(pyrrolidin-1-ylmethyl)phenyl]phenylmethanol (14a)

Starting from 13 (0.60 g, 1.43 mmol) and bromobenzene (0.22 mL, 2.15 mmol), the title compound was prepared using magnesium turnings (52.2 mg, 2.15 mmol) as described for 3a, and was obtained as a light yellow oil (0.57 g, 79%); ¹H NMR (300 MHz, CDCl₃) δ 7.37-7.20 (m, 9H), 7.09-7.06 (m, 2H), 3.96 (bs, 1H), 3.70 (s, 4H), 2.54 (m, 8H), 1.77 (m, 8H); ESI MS m/z 495 (M+H)⁺.

### (±)-(4-Chlorophenyl)[4-(pyrrolidin-1-ylmethyl)phenyl]phenylmethanol (14b)

Starting from 5f (1.25 g, 4.2 mmol) and bromobenzene (4.2 mmol), the title compound was prepared using magnesium turnings (4.2 mmol) as described for 3a, and was obtained as a brown oil (1.20 g, 76%); ¹H NMR (300 MHz, CDCl₃) δ 7.29-7.16 (m, 13H), 3.89 (bs, 1H), 3.58 (s, 2H), 2.49 (m, 4H), 1.73 (m, 4H); ESI MS m/z 378 (M+H)⁺.

### (±)-(4-Chlorophenyl)(4-fluorophenyl)[4-(pyrrolidin-1-ylmethyl)phenyl]methanol (14c)

Starting from 5f (1.10 g, 3.7 mmol) and 1-bromo-4-fluorobenzene (3.7 mmol), the title compound was prepared using magnesium turnings (3.7 mmol) as described for 3a and was obtained as a brown oil (0.94 g, 65%); ¹H NMR (300 MHz, CDCl₃) δ 7.33-7.16 (m, 10H), 7.01-6.99 (m, 2H), 3.66 (s, 2H), 2.78 (bs, 1H), 2.58 (m, 4H), 1.83-1.82 (m, 4H); ESI MS m/z 396 (M+H)⁺.

### (±)-7-Chloro-N-{(4-chlorophenyl)[4-(pyrrolidin-1-ylmethyl)phenyl]phenyl methyl}-4-aminoquinoline (15a)

To a solution of 14b (320 mg, 0.85 mmol) and a drop of N,N-DMF in dry DCM (15 mL), a solution of SOCl₂ (180 μL, 2.54 mmol) in dry DCM (10 mL) was added at 0° C., and the mixture was stirred at 0° C. for 20 min, thereafter was heated to 45° C. for 4 h. The volatiles were removed and the residue was treated with dry DCM (3×10 mL) and concentrated under reduced pressure in order to remove residual SOCl₂. The resulting hydrochloride salt was dissolved in THF (15 mL) and added to a refluxing mixture of sodium hydride (60% in mineral oil, 68.0 mg, 1.7 mmol) and 4-amino-7-chloroquinoline (0.31 mg, 1.7 mmol) in dry THF (20 mL). The resulting mixture was heated under reflux overnight and then was allowed to cool to rt. Subsequently, the reaction mixture was quenched with ice and the aqueous phase was extracted with chloroform. The organic extracts were washed with brine, dried and concentrated in vacuo. The residue was purified by flash-chromathography eluting with a 10% mixture of MeOH in chloroform to afford 15a as a pale yellow oil (105 mg, 23%); ¹H NMR (300 MHz, CDCl₃) δ 8.14 (d, 1H, J=5.2 Hz), 7.95 (d, 1H, 2.3 Hz), 7.74 (d, 1H, J=8.8 Hz), 7.40-7.26 (m, 14H), 6.24 (bs, 1H), 5.86 (d, 1H, J=5.7 Hz), 3.71 (s, 2H), 2.67-2.65 (m, 4H), 1.88-1.83 (m, 4H).

### (±)-7-Chloro-N-{(4-chlorophenyl)(4-fluorophenyl)[4-(pyrrolidin-1-ylmethyl)phenyl]methyl}-4-aminoquinoline (15b)

Starting from 14c and 4-amino-7-chloroquinoline, the title compound was prepared following the procedure described for 15a and was obtained as a brown oil; ¹H NMR (300 MHz, CDCl₃) δ 8.16 (d, 1H, J=5.6 Hz), 7.96 (d, 1H, J=2.1 Hz), 7.72 (d, 1H, J=9.1 Hz), 7.41 (dd, 1H, J=2.1, 8.8 Hz), 7.32-7.20 (m, 10H), 7.03-6.97 (m, 2H), 6.19 (bs, 1H), 5.84 (d, 1H, J=5.6 Hz), 3.60 (s, 2H), 2.53-2.50 (m, 4H), 1.82-1.80 (m, 4H).

### (±)-N-{(4-Chlorophenyl)[4-(pyrrolidin-1-ylmethyl)phenyl]phenylmethyl}-6-chloro-2-methoxy-9-aminoacridine (15c)

Starting from 14a (0.13 g, 0.31 mmol), and 6-chloro-2-methoxyacridin-9-amine (90 mg, 0.35 mmol) in dry DMF, the title compound was prepared following the procedure described for 15a and was obtained as a yellow oil (80 mg, 38%); ¹H NMR (300 MHz, CDCl₃) δ 7.91 (d, 1H, J=2.0 Hz), 7.83 (d, 1H, J=9.3 Hz), 7.50 (d, 1H, J=9.3 Hz), 5.35-7.32 (m, 4H), 7.23-7.17 (m, 5H), 7.12-7.05 (m, 4H), 7.98-6.92 (m, 2H), 6.75 (d, 1H, J=2.3 Hz), 5.40 (s, 1H), 3.46 (s, 2H), 3.38 (s, 3H), 2.32 (m, 4H), 1.74 (m, 4H); ESI MS m/z 618 (M+H)⁺.

### N-{bis[3-Chloro-4-(pyrrolidin-1-ylmethyl)phenyl]phenylmethyl}-7-chloro-4-aminoquinoline (15d)

Starting from 14a (0.16 g, 0.33 mmol), and 4-amino-7-chloroquinoline (70 mg, 0.39 mmol), the title compound was prepared following the above described procedure and was obtained as a yellow oil (23 mg, 11%); ¹H NMR (300 MHz, CDCl₃) δ 8.17 (d, 1H, J=5.2 Hz), 7.97 (d, 1H, J=2.0 Hz), 7.74 (d, 1H, J=9.0 Hz), 7.44-7.40 (m, 3H), 7.32-7.31 (m, 7H), 7.19-7.15 (m, 2H), 6.17 (s, 1H), 5.88 (d, 1H, J=5.5 Hz), 3.72 (s, 4H), 2.57 (m, 8H), 1.79 (m, 8H); ESI MS m/z 657 (M+H)⁺.

### Example 3

### In Vitro Activity of Selected Compounds

Some of the synthesized compounds were tested in vitro against two different strains of P. falciparum, namely 3D7, NF54 and D10 (CQ-S strain) and K1 and W2 (CQ-R strain). The pharmacological results are displayed in Table 2. In this table the values for CLT (clotrimazole) and CQ (chloroquine) are also reported.

TABLE 2

| | | | | | | TC₅₀ (μM) | | | |
|---|---|---|---|---|---|---|---|---|---|
| | IC₅₀ (nM) | | | | | KB | NSO | Daudi | Normal human |
| Cpd | D10^{a} | W2^{b} | 3D7^{a} | K1^{b} | NF54^{a} | cell^{c} | cells^{d} | cells^{e} | lymphocytes^{f} |
| 9a | 20.7 | 22.4 | 24.5 | 65 | 162 | n.t. | 23.81 | 34.63 | 54.11 |
| 9b | 141.9 | 219.6 | n.t.^{g} | 259.2 | 154.7 | n.t. | 44.73 | 43.90 | 40.76 |
| 9c | n.t. | n.t. | n.t. | 19 | 13 | n.t. | n.t. | n.t. | n.t. |
| 9d | n.t. | n.t. | n.t. | 107 | 47 | n.t. | n.t. | n.t. | n.t. |
| 9e | 45.78 | 72.12 | 3.7 | 9 | 15 | 19.61 | 8.65 | 8.65 | 17.30 |
| 9f | n.t. | n.t. | n.t. | 504. | 337 | n.t. | n.t. | n.t. | n.t. |
| 9g | n.t. | n.t. | n.t. | 22 | 17 | n.t. | 1.68 | 1.47 | 1.42 |
| 9h | n.t. | n.t. | n.t. | 20 | 13 | n.t. | n.t. | n.t. | n.t. |
| 9j | n.t. | n.t. | n.t. | 375 | 163 | n.t. | n.t. | n.t. | n.t. |
| 9k | 61.48 | 90.47 | 0.2 | 30 | 20 | 5.49 | 12.10 | 14.11 | 24.20 |
| 9l | n.t. | n.t. | n.t. | 33 | 19.8 | n.t. | n.t. | n.t. | n.t. |
| 9m | n.t. | n.t. | n.t. | 65.5 | 39.3 | n.t. | n.t. | n.t. | n.t. |
| 9n | n.t. | n.t. | n.t. | 67 | 43 | n.t. | n.t. | n.t. | n.t. |
| 9o | n.t. | n.t. | n.t. | 34 | 27 | n.t. | n.t. | n.t. | n.t. |
| 9p | 57.18 | 81.90 | 8 | 60 | 32 | 8.5 | n.t. | n.t. | n.t. |
| 9q | n.t. | n.t. | n.t. | 54 | 33 | n.t. | n.t. | n.t. | n.t. |
| 9r | n.t. | n.t. | n.t. | 58 | 21 | n.t. | n.t. | n.t. | n.t. |
| 9s | 48.65 | 58.76 | 1 | 8 | 167 | n.t. | 11.06 | 11.06 | 16.59 |
| 15a | n.t. | n.t. | 3 | 22 | n.t. | n.t. | 14.86 | 16.71 | 18.57 |
| CLT | 550 | 490 | 60 | 250 | n.t. | 88.1 | n.t. | n.t. | n.t. |
| CQ | 22 | 280 | 10 | 260 | 7.0 | 207.0 | n.t. | n.t. | n.t. |

^{a}CQ-S clone.
^{b}CQ-R clone.
^{c}Human carcinoma of the nasopharynx cell line.
^{d}Plasmocytoma murine cell line.
^{e}Human lymphoblastoid cell line.
^{f}Normal human lymphocytes PHA-stimulated.
^{g}n.t. = not tested.

### Example 4

### In Vivo Activity of 9a,e,k,s and 15a

Compounds 9a,e,k,s and 15a were tested in vivo against P. chabaudi and CLT and CQ were used as reference drugs. The experiment was performed at a single dose of 50 mg/Kg. ED₅₀ was determined for 9a in P. berghei ANKA. The results are shown in Table 3. All compounds were adminstered per os.

TABLE 3

| | P. Chabaudi AS | P. Berghei ANKA |
|---|---|---|
| Compound | (% inhibition at 50 mg/Kg) | (ED₅₀, mg/Kg) |
| 9a | 98.0 | 6.3^{a} |
| 9e | 99.5 | n.t. |
| 9k | 99.6 | n.t. |
| 9s | 99.9 | n.t. |
| 15a | 99.9 | n.t. |
| CLT | 6.0 | — |
| CQ | 100 (10 mg/Kg) | — |

^{a}98% inhibition of parasitemia at 50 mg/Kg per os in P. berghei.

### Example 5

### Antifungal Activity of 9a,e,k,p

We tested yeast and filamentous fungi from international collection (Candida krusei ATCC® 6258, Candida parapsilosis ATCC® 22019 [both quality control strains], Aspergillus flavus ATCC® 204304 [reference strain]) and the following clinical isolates (Candida albicans MGMI1, Candida glabrata MGMI2, Cryptococcus neoformans MGMI3 and two Aspergillus fumigatus MGMI4 e MGMI5, one (MGMI5) itraconazole (ITRA) resistant (MIC>16 mcg/ml)) against compounds 7a-d using the CLSI protocols for yeast and filamentous fungi described in

(1) National Committee for Clinical Laboratory Standards. (2002). Reference method for broth dilution antifungal susceptibility testing of yeasts—Second Edition: Approved Standard, M27-A2. NCCLS, Wayne, Pa.; and

(2) National Committee for Clinical Laboratory Standards. Reference method for broth dilution antifungal susceptibility testing of filamentous fungi: Approved Standard, M38-A. 2002; NCCLS, Wayne, Pa.

The results are shown in Table 4.

TABLE 4

| Strains | 9a | 9e | 9k | 9p | ITRA | CLT |
|---|---|---|---|---|---|---|
| Candida albicans MGMI 1 | >32 | >32 | 16 | >32 | 0.25 | 0.03 |
| Candida glabrata MGMI 2 | >32 | >32 | >32 | >32 | 32 | NT |
| Candida krusei ATCC ® 6258 | >32 | >32 | >32 | >32 | 0.25 | 2 |
| Candida parapsilosis ATCC ® 22019 | 32 | 16 | 16 | >32 | 0.125 | 2 |
| Cryptococcus neoformans MGMI 3 | 16 | 16 | 16 | >32 | 0.25 | 2 |
| Aspergillus flavus ATCC ® 204304 | >32 | >32 | >32 | >32 | 0.06 | 4 |
| Aspergillus fumigatus MGMI 4 | >32 | >32 | >32 | >32 | 0.06 | 4 |
| Aspergillus fumigatus MGMI 5 | >32 | >32 | >32 | >32 | >32 | 4 |

The lack of anti-fungal activity of 9a,e,k,p demonstrated that they selectively interact with free heme and not with heme as a prosthetic group in cythocromes. In fact, inhibition of cythocromes P450 through interaction with the heme prosthetic group is the mechanism of action of antifungal activity of CLT and related azoles.

### Example 6

### β-Hematin Inhibitory Activity

The β-hematin inhibitory activity of compounds 9a, CLT (2) and CQ (1) were determined as described by Parapini et Al (Exp. Parasitol. 2000, 96, 249) by using the BHIA (β-hematin inhibitory activity) assay (hemin in dimethyl sulfoxide-acetate buffer at pH 5.0, 37° C., 18 h) and are presented in Table 5, below.

TABLE 5

| Compound | IC₅₀* |
|---|---|
| 9a | 0.785 |
| CLT | 2.50 |
| CQ | 0.91 ± 0.23 |
| Amodiaquine | 0.79 ± 0.01 |

*Molar equivalents of compound relative to hemin.

IC₅₀ are the mean of at least three determinations. Standard errors were all within 10% of the mean.

1-16. (canceled)

17. A 4-amino-quinoline derivative represented by Formula I, 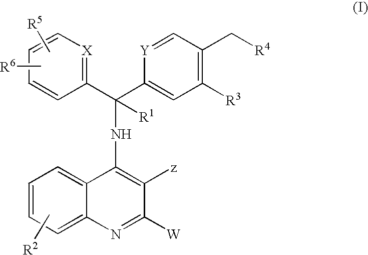
an isomer thereof or a mixture of its isomers, or a pharmaceutically acceptable salt thereof, wherein
X and Y both represent CH; or
X and Y, together with the carbon atoms to which they are attached, form a bridge selected from C—C, C—CH₂—CH₂—C and C—CH═CH—C;
W and Z both represent hydrogen; or
W and Z, together with the carbon atoms to which they are attached, form a benzo-fused ring —CH═CH—CH═CH—, which fused ring may optionally be substituted with halo;
R¹ represents hydrogen, phenyl, halo-substituted phenyl, 3,4-methylendioxyphenyl or (pyrrolidinylmethyl)phenyl;
R² represents halo, trifluoromethyl or alkoxy;
R³ represents hydrogen, halo, hydroxy, cyano, sulfonamido or dialkylsulfonamido;
R⁴ represents N,N-dialkyl-amino, pyrrolidinyl, piperazinyl, morpholinyl or imidazolyl;
R⁵ represents hydrogen, halo, cyano, hydroxy or —SO₂NH₂; and
R⁶ represents hydrogen, N,N-dialkyl-amino-methyl, pyrrolidinyl-methyl, piperazinyl-methyl, morpholinyl-methyl or 1H-imidazolyl-methyl.

18. The 4-amino-quinoline derivative of claim 17, an isomer thereof or a mixture of its isomers, or a pharmaceutically acceptable salt thereof, wherein X and Y both represent CH.

19. The 4-amino-quinoline derivative of claim 17, an isomer thereof or a mixture of its isomers, or a pharmaceutically acceptable salt thereof, wherein X and Y, together with the carbon atoms to which they are attached, form a bridge selected from C—C, C—CH₂—CH₂—C and C—CH═CH—C.

20. The 4-amino-quinoline derivative of claim 17, an isomer thereof or a mixture of its isomers, or a pharmaceutically acceptable salt thereof, wherein W and Z both represent hydrogen.

21. The 4-amino-quinoline derivative of claim 17, an isomer thereof or a mixture of its isomers, or a pharmaceutically acceptable salt thereof, wherein W and Z, together with the carbon atoms to which they are attached, form a benzo-fused ring —CH═CH—CH═CH—, which fused ring may optionally be substituted with halo.

22. The 4-amino-quinoline derivative of claim 17, an isomer thereof or a mixture of its isomers, or a pharmaceutically acceptable salt thereof, wherein W and Z, together with the carbon atoms to which they are attached, form a benzo-fused ring selected from —CH═CH—CH═CH— and —CH═CH—C(Cl)═CH—.

23. The 4-amino-quinoline derivative of claim 17, an isomer thereof or a mixture of its isomers, or a pharmaceutically acceptable salt thereof, wherein
X and Y both represent CH;
W and Z both represent hydrogen;
R₁ represents hydrogen, phenyl, fluorophenyl, 3,4-methylendioxyphenyl or (pyrrolidinylmethyl)phenyl;
R₂ represents halo, trifluoromethyl or alkoxy;
R₃ represents hydrogen, halo, hydroxy, cyano, sulfonamido or dialkylsulfonamido;
R₄ represents N,N-dialkyl-amino, pyrrolidinyl, piperazynyl, morpholinyl or imidazolyl;
R₅ represents hydrogen, halo, cyano, hydroxy or SO₂NH₂; and
R₆ represents hydrogen, N,N-dialkyl-aminomethyl, pyrrolidinylmethyl, piperazynylmethyl, morpholinylmethyl or imidazolylmethyl.

24. The 4-amino-quinoline derivative of claim 17, an isomer thereof or a mixture of its isomers, or a pharmaceutically acceptable salt thereof, wherein
X and Y both represent CH;
W and Z, together with the carbon atoms to which they are attached, form a benzo-fused ring —CH═CH—CH═CH—, which fused ring may optionally be substituted with halo;
R₁ represents hydrogen, phenyl, fluorophenyl, 3,4-methylendioxyphenyl or (pyrrolidinylmethyl)phenyl;
R₂ represents alkoxy;
R₃ represents hydrogen, halo, hydroxy, cyano, sulfonamido or dialkylsulfonamido;
R₄ represents N,N-dialkyl-amino, pyrrolidinyl, piperazynyl, morpholinyl or imidazolyl;
R₅ represents hydrogen, halo, cyano, hydroxy or SO₂NH₂; and
R₆ represents hydrogen, N,N-dialkyl-aminomethyl, pyrrolidinylmethyl, piperazynylmethyl, morpholinylmethyl or imidazolylmethyl.

25. The 4-amino-quinoline derivative of claim 17, an isomer thereof or a mixture of its isomers, or a pharmaceutically acceptable salt thereof, wherein
X and Y, together with the carbon atoms to which they are attached, form a bridge selected from C—C, C—CH₂—CH₂—C and C—CH═CH—C; and
W and Z both represent hydrogen; or
W and Z, together with the carbon atoms to which they are attached, form a benzo-fused ring —CH═CH—CH═CH—, which fused ring may optionally be substituted with halo;
R₁ represents hydrogen;
R₂ represents halo, trifluoromethyl or alkoxy;
R₃ represents hydrogen, halo, hydroxy, cyano, sulfonamido or dialkylsulfonamido;
R₄ represents N,N-dialkyl-amino, pyrrolidinyl, piperazynyl, morpholinyl or imidazolyl;
R₅ represents hydrogen, halo or hydroxy; and
R₆ represents hydrogen, N,N-dialkyl-aminomethyl, pyrrolidinylmethyl, piperazynylmethyl, morpholinylmethyl or imidazolylmethyl.

26. The 4-amino-quinoline derivative of claim 17, which is
(±)-7-Chloro-N-{(3-chlorophenyl)[4-(pyrrolidin-1-ylmethyl)phenyl]methyl}-4-aminoquinoline;
(±)-7-Chloro-N-{(3-chlorophenyl) [4-(morpholin-4-ylmethyl)phenyl]methyl}-4-aminoquinoline;
(±)-7-Chloro-N-{[3-chloro-4-(pyrrolidin-1-ylmethyl)phenyl](3-chlorophenyl)methyl}-4-aminoquinoline;
(±)-7-Chloro-N-{[3-chloro-4-(morpholin-4-ylmethyl)phenyl](3-chlorophenyl)methyl}-4-aminoquinoline;
(±)-7-Chloro-N-{(4-chlorophenyl)[4-(pyrrolidin-1-ylmethyl)phenyl]methyl}-4-aminoquinoline;
(±)-7-Chloro-N-{(4-chlorophenyl)[4-(morpholin-4-ylmethyl)phenyl]methyl}-4-aminoquinoline;
(±)-7-Chloro-N-{(4-chlorophenyl)[4-(piperazin-1-ylmethyl)phenyl]methyl}-4-aminoquinoline;
(±)-7-Chloro-N-{(4-chlorophenyl) [3-fluoro-4-(pyrrolidin-1-ylmethyl)phenyl]methyl}-4-amino quinoline;
(±)-N-{[3-Chloro-4-(pyrrolidin-1-ylmethyl)phenyl](3-chlorophenyl)methyl}-7-trifluoromethyl-4-aminoquinoline;
(±)-N-{[3-Chloro-4-(morpholin-4-ylmethyl)phenyl](3-chlorophenyl)methyl}-7-trifluoromethyl-4-aminoquinoline;
(±)-N-{(4-Chlorophenyl)[4-(pyrrolidin-1-ylmethyl)phenyl]methyl}-7-trifluoromethyl-4-aminoquinoline;
(±)-N-{(4-Chlorophenyl) [3-fluoro-4-(pyrrolidin-1-ylmethyl)phenyl]methyl}-7-trifluoromethyl-4-aminoquinoline;
(±)-6-Methoxy-N-{(3-chlorophenyl) [4-(pyrrolidin-1-ylmethyl)phenyl]methyl}-4-aminoquinoline;
(±)-N-{(3-Chlorophenyl) [4-(morpholin-4-ylmethyl)phenyl]methyl}-6-methoxy-4-aminoquinoline;
(±)-N-{[3-Chloro-4-(pyrrolidin-1-ylmethyl)phenyl] (3-chlorophenyl)methyl}-6-methoxy-4-aminoquinoline;
(±)-6-Methoxy-N-{(4-chlorophenyl) [4-(pyrrolidin-1-ylmethyl)phenyl]methyl}-4-aminoquinoline;
(±)-N-{(4-Chlorophenyl)[4-(morpholin-4-ylmethyl)phenyl]methyl}-6-methoxy-4-aminoquinoline;
(±)-N-{(4-Chlorophenyl)[4-(piperazin-1-ylmethyl)phenyl]methyl}-6-methoxy-4-aminoquinoline;
(±)-6-Chloro-N-{(4-chlorophenyl) [4-(pyrrolidin-1-ylmethyl)phenyl]methyl}-2-methoxy-9-aminoacridine;
(±)-7-Chloro-N-{[4-(1H-imidazol-1-yl)methylphenyl](4-chlorophenyl)methyl}-4-aminoquinoline;
(±)-7-Chloro-N-{(4-chlorophenyl)[4-(pyrrolidin-1-ylmethyl)phenyl]phenyl methyl}-4-aminoquinoline;
(±)-7-Chloro-N-{(4-chlorophenyl)(4-fluorophenyl)[4-(pyrrolidin-1-ylmethyl)phenyl]methyl}-4-aminoquinoline;
(±)-N-{(4-Chlorophenyl) [4-(pyrrolidin-1-ylmethyl)phenyl]phenylmethyl}-6-chloro-2-methoxy-9-aminoacridine; or
N-{bis[3-Chloro-4-(pyrrolidin-1-ylmethyl)phenyl]phenylmethyl}-7-chloro-4-aminoquinoline;
or a pharmaceutically acceptable addition salt thereof.

27. A pharmaceutical composition comprising a therapeutically effective amount of a 4-amino-quinoline derivative of claim 17, or a pharmaceutically-acceptable addition salt thereof, together with one or more adjuvants, excipients, carriers and/or diluents.

28. A method of treatment, prevention or alleviation of an infectious disease, disorder or condition of a living animal body, including a human, which disorder, disease or condition is caused by a parasite of the genus Plasmodium, which method comprises the step of administering to such a living animal body in need thereof, a therapeutically effective amount of the 4-amino-quinoline derivative according to claim 17, or a pharmaceutically-acceptable addition salt thereof.

29. The method according to claim 28, wherein the disease, disorder or condition is caused by P. falciparum, P. berghei, P. vivax, P. ovale, P. malaria or P. knowlesi.

30. The use according to claim 29, wherein the disease, disorder or condition is malaria.
